(19) United States
(12) Patent Application Publication
(10) Pub. No.: US 2005/0119615 A1
(43) Pub. Date: Jun. 2, 2005

(54) Guidewire for crossing occlusions or stenoses

(75) Inventors: Gerardo V. Noriega, Mountain View, CA (US); Victor Chechelski, Mountain View, CA (US); Rudolfo Sudaria, San Jose, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: NORBORN MEDICAL, INC., Mountain View, CA (US)

(21) Appl. No.: 10/999,457

(22) Filed: Nov. 29, 2004

Related U.S. Application Data
(63) Continuation-in-part of application No. 09/644,201, filed on Aug. 22, 2000, now Pat. No. 6,824,550.
(60) Provisional application No. 60/195,154, filed on Apr. 6, 2000.

(51) Int. Cl. A61M 31/00

(52) U.S. Cl. 604/95.04

(57) ABSTRACT

A hollow guidewire for removing tissue from a body lumen, such as a coronary artery. The hollow guidewire comprises an elongate, tubular guidewire body that has an axial lumen. A tissue removal assembly, such as a rotating drive shaft, is positioned at or near a distal end of the tubular guidewire body and extends through the axial lumen. Actuation of the tissue removal assembly removes occlusive material in the body lumen.

### CROSS-REFERENCES TO RELATED APPLICATIONS

The present application is a continuation-in-part of U.S. patent application Ser. No. 09/644,201, entitled “Guidewire for Crossing Occlusions or Stenoses,” (allowed), which claimed benefit under 37 C.F.R. § 1.78 to U.S. Provisional Patent Application No. 60/195,154, filed Apr. 6, 2000, entitled “Guidewire for Crossing Occlusions or Stenosis,” the complete disclosures of which are incorporated herein by reference.

The present application is also related to U.S. patent application Ser. No. 09/030,657, filed Feb. 25, 1998, and U.S. patent application Ser. No. 09/935,534, filed Aug. 22, 2001, now U.S. Pat. No. 6,746,422, entitled “Steerable Support System with External Ribs/Slots that Taper,” the complete disclosure of which are incorporated herein by reference.

### BACKGROUND OF THE INVENTION

The present invention is generally related to medical devices, kits, and methods. More specifically, the present invention provides a guidewire system for crossing stenosis, partial occlusions, or total occlusions in a patient's body.

Cardiovascular disease frequently arises from the accumulation of atheromatous material on the inner walls of vascular lumens, particularly arterial lumens of the coronary and other vasculature, resulting in a condition known as atherosclerosis. Atheromatous and other vascular deposits restrict blood flow and can cause ischemia which, in acute cases, can result in myocardial infarction or a heart attack. Atheromatous deposits can have widely varying properties, with some deposits being relatively soft and others being fibrous and/or calcified. In the latter case, the deposits are frequently referred to as plaque. Atherosclerosis occurs naturally as a result of aging, but may also be aggravated by factors such as diet, hypertension, heredity, vascular injury, and the like.

Atherosclerosis can be treated in a variety of ways, including drugs, bypass surgery, and a variety of catheter-based approaches which rely on intravascular widening or removal of the atheromatous or other material occluding the blood vessel. Particular catheter-based interventions include angioplasty, atherectomy, laser ablation, stenting, and the like. For the most part, the catheters used for these interventions must be introduced over a guidewire, and the guidewire must be placed across the lesion prior to catheter placement. Initial guidewire placement, however, can be difficult or impossible in tortuous regions of the vasculature. Moreover, it can be equally difficult if the lesion is total or near total, i.e. the lesion occludes the blood vessel lumen to such an extent that the guidewire cannot be advanced across the lesion.

To overcome this difficulty, forward-cutting atherectomy catheters have been proposed. Such catheters usually can have a forwardly disposed blade (U.S. Pat. No. 4,926,858) or rotating burr (U.S. Pat. No. 4,445,509). While effective in some cases, these catheter systems, even when being advanced through the body lumen with a separate guidewire, have great difficulty in traversing through the small and tortuous body lumens of the patients and reaching the target site.

For these reasons, it is desired to provide devices, kits, and methods which can access small, tortuous regions of the vasculature and which can remove atheromatous, thrombotic, and other occluding materials from within blood vessels. In particular, it is desired to provide atherectomy systems which can pass through partial occlusions, total occlusions, stenosis, and be able to macerate blood clots or thrombotic material. It is further desirable that the atherectomy system have the ability to infuse and aspirate fluids before, during, or after crossing the lesion. At least some of these objectives will be met by the devices and methods of the present invention described hereinafter and in the claims.

### BRIEF SUMMARY OF THE INVENTION

The present invention provides systems and methods for removing occlusive material and passing through occlusions, stenosis, thrombus, and other material in a body lumen. More particularly, the present invention can be used for passing through stenosis or occlusions in a neuro, cardio, and peripheral body lumens. Generally, the present invention includes an elongate member, such as a hollow guidewire, that is advanced through a body lumen and positioned adjacent the occlusion or stenosis. A tissue removal assembly is positioned at or near a distal tip of the hollow guidewire to create an opening in the occlusion. In exemplary embodiments, the tissue removal assembly comprises a drive shaft having a distal tip that is rotated and advanced from within an axial lumen of the hollow guidewire. Once the guidewire has reached the lesion, the guidewire with the exposed rotating drive shaft may be advanced into the lesion (or the guidewire may be in a fixed position and the drive shaft may be advanced) to create a path forward of the hollow guidewire to form a path in the occlusion or stenosis. To facilitate passing through the occlusion or stenosis, the distal end of the hollow guidewire can be steerable to provide better control of the creation of the path through the occlusion or stenosis. Optionally, the target site can be infused and/or aspirated before, during, and after creation of the path through the occlusion.

The hollow guidewire of the present invention has a flexibility, pushability and torqueability to be advanced through the tortuous blood vessel without the use of a separate guidewire or other guiding element. Additionally, the hollow guidewire may be sized to fit within an axial lumen of a conventional support or access catheter system. The catheter system can be delivered either concurrently with the advancement of the hollow guidewire or after the hollow guidewire or conventional guidewire has reached the target site. The position of the hollow guidewire and catheter system can be maintained and stabilized while the drive shaft is rotated and translated out of the axial lumen of the hollow guidewire. The distal tip of the drive shaft can be deflected, coiled, blunted, flattened, enlarged, twisted, basket shaped, or the like. In some embodiments, to increase the rate of removal of the occlusive material, the distal tip is sharpened or impregnated with an abrasive material such as diamond chips, diamond powder, glass, or the like.

The drive shaft can be a counter-wound guidewire construction or be composed of a composite structure comprising a fine wire around which a coil is wrapped. The counter-wound or composite constructions are more flexible than a single wire drive shaft and can provide a tighter bending radius while still retaining the torque transmitting ability so that it can still operate as a lesion penetration mechanism.

In a specific configuration, the drive shaft has spiral threads or external riflings extending along the shaft. The spirals typically extend from the proximal end of the shaft to a point proximal of the distal tip. As the drive shaft is rotated and axially advanced into the occlusive material (concurrently with the hollow guidewire body or with the hollow guidewire body substantially stationary), the distal tip creates a path through the occlusion and removes the material from the body. The spirals on the shaft act similar to an “Archimedes Screw” and transport the removed material proximally through the axial lumen of the hollow guidewire and prevents the loose atheromatous material from escaping into the blood stream.

Systems and kits of the present invention can include a support system or access system, such as a catheter having a body adapted for intraluminal introduction to the target blood vessel. The dimensions and other physical characteristics of the access system body will vary significantly depending on the body lumen which is to be accessed. In the exemplary case, the body of the support or access system is very flexible and is suitable for introduction over a conventional guidewire or the hollow guidewire of the present invention. The support or access system body can either be for “over-the-wire” introduction or for “rapid exchange,” where the guidewire lumen extends only through a distal portion of the access system body. Optionally, the support or access system can have at least one axial channels extending through the lumen to facilitate infusion and/or aspiration of material from the target site. Support or access system bodies will typically be composed of an organic polymer, such as polyvinylchloride, polyurethanes, polyesters, polytetrafluoroethylenes (PTFE), silicone rubbers, natural rubbers, or the like. Suitable bodies may be formed by extrusion, with one or more lumens that extend axially through the body. For example, the support or access system can be a support catheter, interventional catheter, balloon dilation catheter, atherectomy catheter, rotational catheter, extractional catheter, laser ablation catheter, guiding catheter, stenting catheter, ultrasound catheter, and the like.

In use, the access system can be delivered to the target site over a conventional guidewire. Once the access system has been positioned near the target site, the conventional guidewire can be removed and the elongate member (e.g., hollow guidewire) of the present invention can be advanced through an inner lumen of the access system to the target site. Alternatively, because the elongate member can have the flexibility, pushability, and torqueability to be advanced through the tortuous regions of the vasculature, it is possible to advance the elongate member through the vasculature to the target site without the use of the separate guidewire. In such embodiments, the access system can be advanced over the elongate member to the target site. Once the elongate member has been positioned at the target site, the drive shaft is rotated and advanced into the occlusive material or the entire elongate member may be advanced distally into the occlusion. The rotation of the distal tip creates a path forward of the elongate member. In some embodiments the path created by the distal tip has a path radius which is larger than the radius of the distal end of the elongate member. In other embodiments, the path created by the distal tip has a path radius which is the same size or smaller than the radius of the elongate member.

One exemplary hollow guidewire for crossing an occlusion or stenosis within a body lumen comprises an hollow guidewire body comprising a proximal opening, a distal opening, and an axial lumen extending from the proximal opening to the distal opening. A rotatable drive shaft is disposed within the axial lumen, wherein a distal tip of the rotatable drive shaft is adapted to extend distally through the distal opening in the guidewire body. At least one pull wire extends through the axial lumen and is coupled to a distal end portion of the guidewire body. The pull wire(s) comprise a curved surface that substantially corresponds to a shape of an inner surface of the axial lumen.

In one preferred configuration, the hollow guidewire body is composed of a single, laser edged hypotube. In one configuration, a proximal portion of the hollow guidewire comprises one or more sections that comprise a constant pitch. A distal portion of the hollow guidewire may have at least one section that ha a pitch that decreases in the distal direction so as to increase a flexibility in the distal direction along the distal portion of the guidewire body.

In other configurations, the hollow guidewire body optionally comprises a section that comprises no helical windings and has a solid wall. In other configurations, the distal portion may have a pitch that is constant, or the pitch may increase in the distal direction. In many embodiments, the hollow guidewire body will have at least one section that has a right-handed coils and at least one section that has left handed coils. In preferred configurations, the sections with the right handed coils alternate with the sections that have the left handed coils.

The dimensions of the hollow guidewires of the present invention will vary but the largest radial dimension (e.g., outer diameter) is typically between approximately 0.009 inches and 0.040 inches, preferably between approximately 0.035 inches and approximately 0.009 inches, more preferably between approximately 0.024 inches and 0.009 inches, and most preferably between approximately 0.013 and approximately 0.014 inches. A wall thickness of the hollow guidewires of the present invention is typically between approximately 0.001 inches and approximately 0.004 inches, but as with the other dimensions will vary depending on the desired characteristics of the hollow guidewire. The construction of the hollow guidewire will typically provide a 1:1 torqueability and the hollow guidewire will have the torqueability, pushability, and steerability to be advanced through the body lumen without the need of an additional guidewire or other guiding element.

A distal end portion of the hollow guidewire may comprise a plurality of openings or thinned portions that extend circumferentially or radially about at least a portion of the distal end portion of the guidewire body. A rib or other supporting structure will be disposed between each of the openings so as to provide structural support to the distal end portion. The plurality of openings or thinned portions may be used to increase the flexibility and/or bendability of the distal end portion, such that when the pull wires are actuated, the distal end portion is able to deflect without causing kinking in the distal end portion. The distal end portion may also include one or more radiopaque markers to assist in the fluoroscopic tracking of the hollow guidewire.

The hollow guidewires of the present invention may comprise only a single pull wire. In other embodiments, the hollow guidewire comprises two or more pull wires. The pull wires of the present invention may optionally be coated with Teflon® so as to reduce the friction coefficient of the surface and to reduce twisting of the pull wires. As noted above, the pull wires preferably comprise a curved surface that substantially corresponds to an inner surface of the axial lumen of the hollow guidewire. By providing a surface that substantially corresponds to a shape in the inner surface of the axial lumen, the pull wires are able to move radially outward away from the rotating drive shaft. The increased distance away from the center of the axial lumen provides a greater clearance between the pull wires and the rotating drive shaft, while maintaining a thickness and width of the pull wire.

The pull wires may take on a variety of cross-sectional shapes, but the pull wires typically typically have either a D-shape, crescent shape, or an oval shape. As can be appreciated, other embodiments of the pull wires may have a cross-section that is circular, substantially flattened, substantially rectangular, or the like.

In preferred embodiments, in addition to the curved surface that substantially corresponds to the inner surface of the axial lumen, the pull wires typically comprise a flat surface that is adapted to be adjacent the rotating drive shaft. Since the flat surface of the pull wire will provides only a single point of contact with the rotating drive shaft, there is a reduced friction between the pull wire and the drive shaft and there is a reduced chance that the rotating drive shaft gets tangled with the pull wire.

The rotatable drive shaft of the present invention may be axially movable and rotatable within the axial lumen of the hollow guidewire body. Optionally, the rotatable drive shaft may be coated with Teflon® or other materials to improve the rotation of the drive shaft within the axial lumen. The hollow guidewire may comprise a rotating mechanism, such as a rotary drive motor, to control the rotation of the drive shaft. The rotating mechanism can be coupled to the proximal end of the drive shaft to rotate the drive shaft. Optionally, an actuator may be used to control the axial movement of the drive shaft and/or the rotation of the drive shaft. Activation of the actuator moves the drive shaft proximally and distally within the axial lumen of the hollow guidewire. The hollow guidewire may comprise an additional actuator to control the steering or deflection of a distal portion of the hollow guidewire so as to assist in navigating the hollow guidewire through the body lumen.

The hollow guidewires of the present invention may comprise a removable housing coupled to the proximal portion of the hollow guidewire body. The removable housing may comprise a connector assembly that allows for infusion or aspiration, the actuator(s) (for controlling the rotation, axial movement of the drive shaft and/or steering of the distal end portion of the hollow guidewire body), a rotating member (e.g., drive motor), a control system, and/or a power supply. The removable housing allows for advancement of a catheter system over the hollow guidewire. Once the catheter or other elongate body is advanced over the hollow guidewire, the housing may be reattached so as to allow for actuation of the drive shaft.

In another aspect, the present invention provides a hollow guidewire that comprises a hypotube that comprises a proximal portion and a distal portion. At least a part of the distal portion of the hypotube comprise helical windings formed thereon so that the distal portion of the hypotube is more flexible than the proximal portion. While not described in detail herein, it should be appreciated that in other embodiments, the hollow guidewire may be comprised of a braided polymer, carbon, or other composite materials, and the hollow guidewires of the present invention are not limited to hypotubes.

In such configurations, the proximal portion of the hypotube will have a solid wall or helical windings that have a pitch that is larger than a pitch of the distal portion. Typically, a pitch of the helical windings on the distal portion decreases in the distal direction so that a flexibility of the distal end portion increases in the distal direction. Consequently, the proximal portion is the stiffest, an intermediate portion is less stiff, and the distal end is the most flexible. In other embodiments, the pitch may be constant throughout at least a portion of the distal portion, may increase in the distal direction, the pitch may vary throughout the distal portion, or the like.

The distal portion of the hypotube hollow guidewire may optionally comprise a plurality of ribs and openings or thinned portions that extend circumferentially about at least a portion of the distal end portion of the guidewire body. The distal portion may also comprise one or more radiopaque markers thereon.

Similar to the other embodiments, the hypotube hollow guidewire may comprise one or more pull wires. The pull wires preferably comprise a curved surface that substantially corresponds to an inner surface of the axial lumen of the hypotube hollow guidewire, but other conventional shaped pull wires that don't substantially correspond to the inner surface of the axial lumen may also be used. The pull wire may be coupled to a removable proximal housing that is coupled to the proximal portion of the hypotube hollow guidewire body. A removable housing may be coupled to the hollow guidewire and may comprise a connector assembly that allows for infusion or aspiration, one of more actuators (for controlling the rotation, axial movement of the drive shaft and/or steering of the distal end portion of the hypotube hollow guidewire body), a rotating member (e.g., drive motor), a control system, and/or a power supply.

In a further aspect, the present invention provides a steerable guidewire comprising a hollow guidewire body that comprises a proximal end, a distal end, and an axial lumen that extends to the distal end. At least a portion of a tissue removal assembly is positioned at or near the distal end of the guidewire body. At least one pull wire extends through the axial lumen of the hollow guidewire body and is coupled at or near the distal end of the hollow guidewire body. A proximal force on the pull wire steers the distal end of the hollow guidewire.

The tissue removal assembly may be fixedly or movably disposed at the distal end of the hollow guidewire body. If the tissue removal assembly is movable, the tissue removal assembly may be movable from a first, axially retraced position in which the tissue removal assembly is disposed within the axial lumen of the hollow guidewire body to a second position in which the tissue removal assembly is positioned beyond the distal end of the guidewire body.

The tissue removal assembly typically comprises a rotatable drive shaft that has a shaped distal tip. In other embodiments, however, the tissue removal assembly may comprise a laser, an RF electrode, a heating element (e.g., resistive element), an ultrasound transducer, or the like. A lead of the tissue removal assembly may extend from proximally through an axial lumen of the hollow guidewire body.

In one preferred configuration, the hollow guidewire body is composed of a single hypotube. The hollow guidewire body optionally comprises a helical coil or solid wall tubular proximal portion integrally formed with the distal end portion. The distal end portion may comprise helical windings formed thereon. A pitch between adjacent helical windings on the distal portion decreases in the distal direction so as to increase a flexibility in the distal direction along the distal portion of the guidewire body. In other embodiments, the distal portion may have one or more sections that have a pitch that is constant throughout the distal portion, a pitch that increases in the distal direction, or the like.

A distal end portion of the hollow guidewire may comprise a plurality support ribs and openings or thinned portions that extend circumferentially about at least a portion of the distal end portion of the guidewire body. The plurality of openings or thinned portions may be used to increase the flexibility and/or bendability of the distal end portion, such that when the pull wires are actuated, the distal end portion is able to deflect without kinking of the distal end portion. The distal end portion may also include one or more radiopaque markers to assist in the fluoroscopic tracking of the hollow guidewire.

Similar to the other embodiments, the hollow guidewire may comprise one or more pull wires. The pull wires preferably comprise a curved surface that substantially corresponds to an inner surface of the axial lumen of the hollow guidewire, but other conventional shaped pull wires that don't substantially correspond to the inner surface of the axial lumen may also be used. The pull wire may be coupled to a removable proximal housing that is coupled to the proximal portion of the hollow guidewire body. The removable housing may comprise a connector assembly that allows for infusion or aspiration, one of more actuators (for controlling the rotation, axial movement of the drive shaft and/or steering of the distal end portion of the hollow guidewire body), a rotating member (e.g., drive motor), a control system, and/or a power supply.

In yet another aspect, the present invention provides a hollow guidewire that comprises a proximal portion and a distal portion. At least a part of the distal portion comprises helical windings that have a pitch between adjacent windings that decreases in the distal direction so that a distal end of the hollow guidewire is more flexible than the proximal portion of the hollow guidewire.

In yet another aspect, the present invention provides a method of crossing an occlusion or stenosis within a body lumen. The method comprises positioning an hollow guidewire having a drive shaft in the body lumen. The drive shaft is rotated. The drive shaft is moved from a retracted configuration to an expanded configuration. In the expanded configuration, the drive shaft may be used to create a path that is at least as large as a largest radial dimension (e.g., diameter) of the distal end of the hollow guidewire The hollow guidewire body and/or the drive shaft may then advanced into the occlusion or stenosis to create the path in the occlusion or stenosis.

In another aspect, the present invention provides a method of crossing an occlusion or stenosis within a body lumen. The method comprises advancing a guidewire through the body lumen. An access or support system is moved over the guidewire to the occlusion or stenosis. The guidewire is removed from the body lumen and exchanged with a steerable hollow guidewire having tissue removal assembly. The tissue removal assembly may then be used to remove at least a portion of the occlusion. For example, in one configuration the tissue removal assembly comprises a rotatable drive shaft. The drive shaft is rotated within a lumen of the hollow guidewire and is at least partially exposed through a distal opening in the hollow guidewire. The hollow guidewire and/or the drive shaft may be advanced to create a path through the occlusion or stenosis.

In another aspect, the present invention provides a kit. The kit has any of the hollow guidewire described herein and instructions for use that provide any of the methods described herein. In one configuration, the hollow guidewire comprises a tissue removal assembly, such as a rotatable drive shaft. The rotatable drive shaft has a shaped distal tip that is removably received within the axial lumen of the hollow guidewire. The instructions for use in passing occlusions or stenosis in a body lumen comprise rotating the inner wire within the steerable hollow guidewire and advancing the hollow guidewire and drive shaft or only advancing the rotating drive shaft into the occlusive or stenotic material to create a path through the occlusive or stenotic material. A package is adapted to contain the hollow guidewire, rotatable wire, and the instructions for use. In some embodiments, the instructions can be printed directly on the package, while in other embodiments the instructions can be separate from the package.

These and other aspects of the invention will be further evident from the attached drawings and description of the invention.

### BRIEF DESCRIPTION OF THE DRAWINGS

FIG. 1 shows an elevational view of a system of the present invention;

FIG. 2 shows manual manipulation of a drive shaft of the present invention;

FIG. 3 shows a distal end of the elongate member and a distal tip of a drive shaft of the present invention;

FIG. 3A is a cross sectional view of the device FIG. 3;

FIG. 4 illustrates another embodiment of a hollow guidewire of the present invention.

FIG. 5A is a cross-sectional view of a hollow guidewire that comprises a drive shaft and a flattened or rectangular pull wire.

FIG. 5B is a cross sectional view of a hollow guidewire that comprises a drive shaft and a shaped pull wire.

FIG. 5C is a cross-sectional view of an embodiment that comprises a plurality of spaced, shaped pull wires.

FIG. 6 illustrates another embodiment of a hollow guidewire that includes a plurality of openings or thinned portion in the distal end portion that correspond to the number of pull wires.

FIG. 7 illustrates one exemplary embodiment of a hollow guidewire that comprises left hand coil portions and right hand coil portions, and a coil disposed at the distal tip.

FIG. 7A to 7C are cross sectional views at A-A, B-B, and C-C of a distal portion of the hollow guidewire of FIG. 7, respectively.

FIGS. 8A and 8B are helical coils that have a similar pitch but a different kerf.

FIG. 9 illustrates embodiment of a hollow guidewire that comprises a window formed in the distal portion of the hollow guidewire.

FIG. 9A to 9C are cross sectional views at A-A, B-B, and C-C of the distal portion of the hollow guidewire of FIG. 9, respectively.

FIG. 10 shows a diamond chip embedded distal tip of the drive shaft;

FIG. 11A shows a deflected distal tip in a position forward of the distal end of the elongate member;

FIG. 11B shows the flexible deflected distal tip in a fully retracted position within the axial lumen of the elongate member;

FIG. 11C shows a deflected distal tip in a retracted position with the distal tip partially extending out of the elongate member;

FIG. 12A shows a sharpened deflected distal tip extending out of the elongate member;

FIGS. 12B and 12C show the cutting edges on the deflected distal tip of FIG. 12A;

FIG. 12D shows the distal tip deflected off of the longitudinal axis of the drive shaft;

FIGS. 12E and 12F is a partial cut away section of two counter-wound drive shafts of the present invention;

FIG. 12G shows the relative flexibility between a conventional drive shaft and a counter-wound drive shaft of the present invention;

FIGS. 13A to 13C illustrate a method of forming the deflected distal tip using a fixture;

FIGS. 14A-14K show a variety of tip configurations;

FIG. 14L shows a distal tip having a flattened and twisted configuration;

FIGS. 14M-14P show an exemplary method of manufacturing the distal tip of FIG. 14L;

FIG. 15 shows a drive shaft having spirals or external riflings which facilitate the proximal movement of the removed occlusive or stenotic material;

FIG. 16 shows a linkage assembly between the motor shaft and the drive shaft;

FIGS. 17A and 17B show an alternative linkage assembly coupling the motor shaft and the drive shaft;

FIGS. 18-20 show a luer connection assembly which couples the elongate member to the housing;

FIG. 21 shows a system having an access system, a hollow guidewire with a deflectable distal end, and a drive shaft;

FIGS. 22A to 22E illustrate a method of the present invention;

FIGS. 23A to 23E illustrate another method of the present invention;

FIGS. 24A to 24B illustrate yet another method of the present invention; and

FIG. 25 shows a kit of the present invention.

### DETAILED DESCRIPTION OF THE INVENTION

The systems, devices and methods according to the present invention will generally be adapted for the intraluminal treatment of a target site within a body lumen of a patient, usually in a coronary artery or peripheral blood vessel which is occluded or stenosed with atherosclerotic, stenotic, thrombotic, or other occlusive material. The systems, devices and methods, however, are also suitable for treating stenoses of the body lumens and other hyperplastic and neoplastic conditions in other body lumens, such as the ureter, the biliary duct, respiratory passages, the pancreatic duct, the lymphatic duct, and the like. Neoplastic cell growth will often occur as a result of a tumor surrounding and intruding into a body lumen. Removal of such material can thus be beneficial to maintain patency of the body lumen. While the remaining discussion is directed at passing through atheromatous or thrombotic occlusive material in a coronary artery, it will be appreciated that the systems and methods of the present invention can be used to remove and/or pass through a variety of occlusive, stenotic, or hyperplastic material in a variety of body lumens.

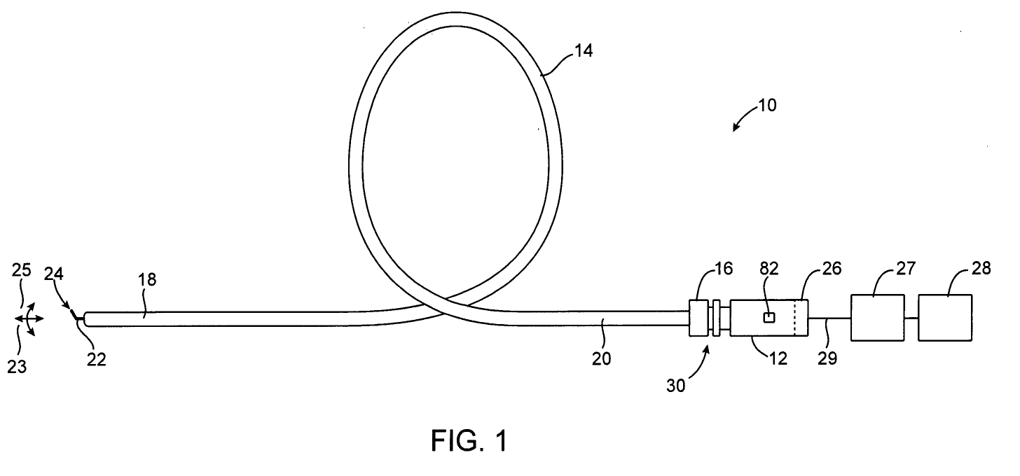

An apparatus 10 embodying features of the present invention is illustrated in FIG. 1. The apparatus 10 generally includes a housing 12 coupled to an elongate member 14 which has a proximal end 16, a distal end 18, and an axial lumen 20 therethrough. The apparatus may comprise a tissue removal assembly, such as a rotatable drive shaft 22, for removing tissue and creating a path through the body lumen. The drive shaft 22 is movably received within the axial lumen 20 of the elongate member 14 and may be rotated and moved axially (as shown by arrows 23, 25). The distal tip 24 of the drive shaft 22 may have a shaped profile such that movement or positioning of the distal tip 24 beyond the distal end 18 of the elongate member and rotation of the drive shaft 22 may be used to create a cutting path forward of the distal end of the elongate member 14 for passing through the occlusive or stenotic material in the body lumen. In most configurations, wire leads 29 couple a drive motor 26 to a control system 27 and a power supply 28. In some embodiments, the power supply 28 is covered with a plastic sheath cover (not shown) so as to maintain a sterile environment.

The drive motor 26 is attachable to a proximal end of the drive shaft 22 to move (i.e., rotate, translate, reciprocate, vibrate, or the like) the drive shaft 22 and shaped distal tip 24. An actuator or input device 82 is attached to the housing 12 to actuate the movement (e.g., control the rotation and/or axial movement) of the drive shaft 22. While not shown, an additional actuator or input device may be attached to housing 12 to control the deflection of a distal portion of the elongate member 14. The proximal end 16 of elongate member 14 is coupled to the housing 12 through a connector assembly 30. The connector assembly 30 limits the motion of the elongate member 14 while allowing the drive shaft 22 to rotate and translate within the elongate member 14. Optionally, some embodiments of the connector assembly 30 includes an aspiration or infusion port (not shown) for facilitating fluid exchange (e.g., delivery or removal) at the target site through the axial lumen 20.

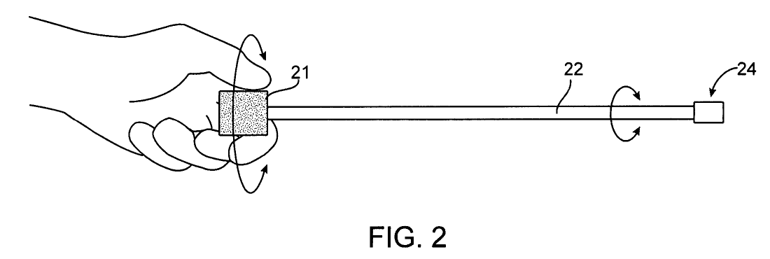

As shown in FIG. 2, in order to macerate clots and to penetrate soft lesions, some drive shafts 22 of the present invention can be configured to be manually rotated. In such embodiments, the proximal end of the drive shaft 22 can be grasped between the fingers and manually turned to rotate the distal tip 24 (shown schematically as a box). The proximal end can be optionally fit with a knurled knob 21 or other mechanism which allows manual manipulation of the proximal end of the drive shaft 22.

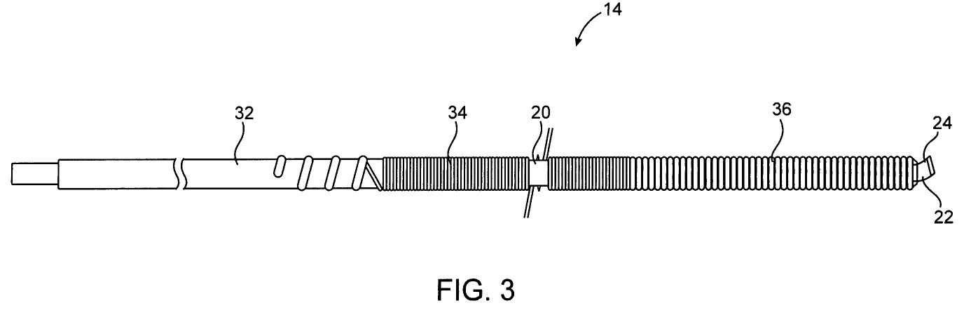

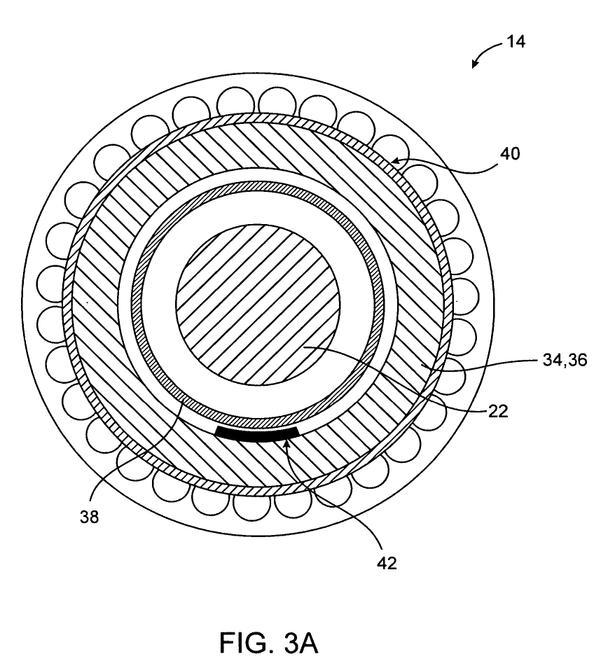

An exemplary embodiment of the elongate member 14 is best seen in FIGS. 3 to 9C. The elongate member 14 is preferably a flexible, hollow guidewire that has the flexibility, pushability, and torqueability to allow a user to advance the hollow guidewire directly through a tortuous blood vessel to the target site. Because of the high columnar strength of the hollow guidewire 14 there is typically no need for a separate guidewire to advance the hollow guidewire 14 to the lesion at the target site.

In the exemplary embodiment illustrated in FIG. 3, the hollow guidewire has an helically wound elongated shaft which defines the axial lumen 20 that receives the drive shaft 22. The axial lumen 20 may further be used for infusion or aspiration. The hollow guidewire 14 includes a proximal tube 32, an intermediate coil 34, and a distal coil tip 36. In some embodiments the intermediate coil 34 is made of a stainless steel or nitinol coil, while the distal coil tip 36 is composed of a flexible, radiopaque coil, such as platinum-iridium. As shown in FIG. 3, the intermediate coil 34 may be threadedly engaged with the proximal tube 32 and threadedly engaged with the distal tip 36. It will be appreciated, however, that the intermediate coil 34 can be connected to the proximal tube 32 and distal coil tip 36 by any conventional means, e.g. solder, adhesive, or the like. The proximal tube 32 of the hollow guidewire 14 can be coupled to a vacuum source or a fluid source (not shown) such that the target site can be aspirated or infused during the procedure, if desired.

Hollow guidewire 14 is typically sized to be inserted through coronary, neuro, or peripheral arteries and can have a variety of diameters. The largest radial dimension (e.g., outer diameter) of the hollow guidewire is typically between approximately 0.009 inches and 0.040 inches, preferably between approximately 0.009 inches and 0.035 inches, and more preferably between approximately 0.009 inches and 0.024 inches, and most preferably between about 0.013 inches and approximately 0.014 inches so as to ensure compatibility with existing interventional cardiology catheters and stent systems. The length of the hollow guidewire 14 may be varied to correspond to the distance between the percutaneous access site and the target site, but is typically about five feet in length. For example, for a target site within the heart that is being accessed through the femoral artery, the hollow guidewire will typically have a length of approximately 175 cm. It should be noted however, that other embodiments of the hollow guidewire 14 may have dimensions that are larger or smaller than the above described embodiments and the present invention is not limited to the above recited dimensions.

Referring now to FIG. 3A, a cross section of the embodiment of FIG. 3 is shown. An inner tube 38 and outer tube 40 are positioned around intermediate coil and distal coil tip 34, 36 to provide a flexible, structural support which prevents liquids from moving between the blood vessel and the axial lumen of the elongate member 14. A reinforcing pull wire 42 can be positioned between the inner tube 38 and the coils 34, 36 to provide for deflection or steering of the distal end 18. The reinforcing pull wire 42 can be formed of a material having sufficient strength so that a thin profile is possible. For example, the reinforcing wire can be an at least partially flattened strip of stainless steel that can retain its shape until it is re-shaped to a different configuration. In one configuration, the reinforcing pull wire 42 is soldered or otherwise connected to the distal end of coil tip 36 and the remainder of the reinforcing pull wire 42 extends proximally through axial lumen 20 to the housing 12. Manipulation of an actuator or the proximal end of the reinforcing pull wire 42 that causes axial movement of the pull wire 42 allows the user to deflect or steer the distal end 18 without permanently impairing the inner structure of the hollow guidewire 14. The steerable distal end 18 provides a user with greater intraluminal control of removing the occlusive or stenotic material from the blood vessel and also aids in navigating the hollow guidewire to the target site. In another configuration, the reinforcing pull wire is 42 can be soldered or otherwise connected to both the distal end and to the junction between coils 34, 36. Therefore, if the coils 34, 36, break, the attached reinforcing pull wire 42 can prevent the coils 34, 36 from detaching from the apparatus 10. A more complete description of one hollow guidewire encompassed by the present invention can be found in commonly owned U.S. patent application Ser. No. 09/030,657, filed Feb. 25, 1998, the complete disclosure of which was previously incorporated by reference.

FIG. 4 illustrates another embodiment of a hollow guidewire 14 that is encompassed by the present invention. In the embodiment of FIG. 4, the hollow guidewire 14 is composed of a single hypotube 37. A radiopaque marker 33 may be disposed on the distal portion 39 of the hypotube 37, and typically at the distal tip. At least the distal portion 39 of the hypotube 37 may be laser edged to create a plurality of helical windings or spirals 43. The helical windings 43 may have the same pitch through at least one section of the distal portion 39 (not shown) or the helical windings 43 may have a variable pitch through at least one section of distal portion 39. As can be appreciated, the pitch between adjacent windings will affect the flexibility of hypotube 37, and the pitch may be selected by the manufacturer depending on the desired characteristics of the hollow guidewire body 14. Because of the flexible nature of the present invention, the manufacturer may provide different configurations of the hollow guidewire so as to enhance the performance (e.g., provide personalized levels of torque response, flexibility, and deflection) of the guidewire body for the specific procedure.

In one configuration, the pitch between the helical windings 43 decreases in the distal direction so as to be increasingly flexible in the distal direction. Consequently, the distal portion 39 of the hypotube 37 will have an increasing flexibility in the distal direction. Advantageously, because the distal portion 39 is integrally formed with the proximal portion 45, there are no joints and there is an improved reliability and a reduced chance of disengagement between the distal portion 39 and the proximal portion 45. It may be desirable to have sections of the guidewire body to have no helical cuts, or to have laser cuts that have a pitch that increases in the distal direction so as to provide less flexibility over a portion of the hollow guidewire. The less flexible portion may be at the proximal portion, an intermediate portion, at or near the distal end of the hollow guidewire, or any combination thereof. For example, in one configuration, a proximal portion 45 of the hypotube may optionally have a solid wall with no laser cuts or helical spirals, and the remainder of the hypotube may have a helical laser edging (which may or may not have a decreasing pitch in the distal direction).

The laser cuts may extend all the way from the proximal end to the distal tip or the laser cuts may extend through less than all of the hypotube. The laser cuts used to create the helical windings may extend completely through the wall of the hypotube or it may extend only partially through the hypotube wall so as to create thinner wall portions (e.g., grooves).

Because the embodiment of FIG. 4 is composed of a single hypotube, there is a no need for the inner and outer support tubes 38, 40. Consequently, the effective outer diameter of the hypotube may be reduced and the diameter or the inner axial lumen 20 will be effectively increased to accommodate a larger drive shaft or pull wire(s) 42.

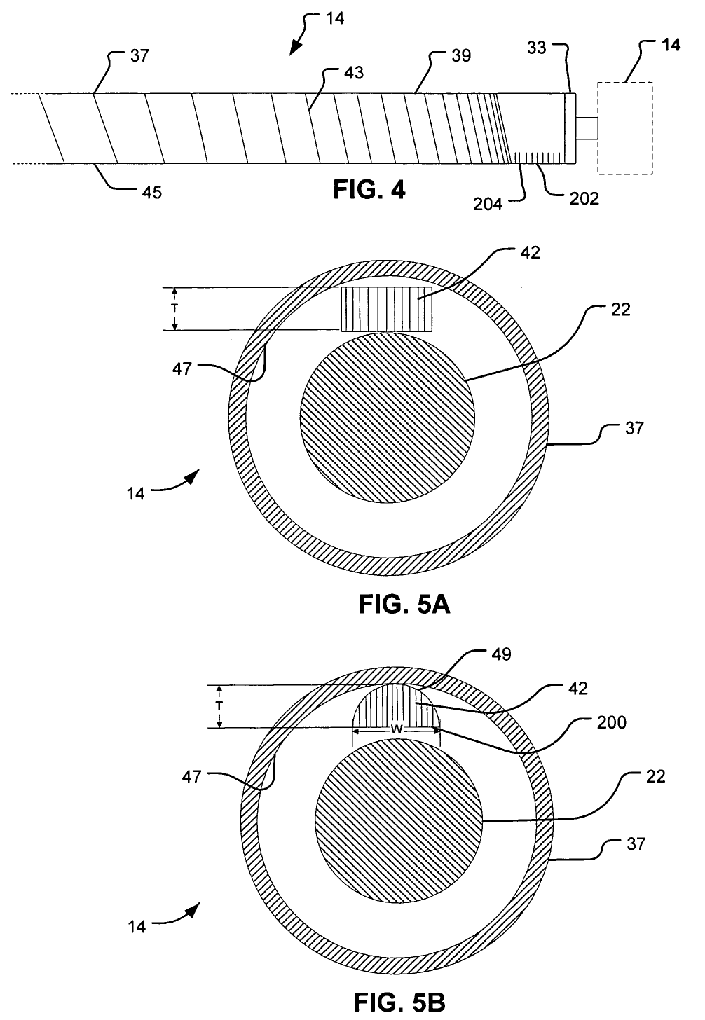

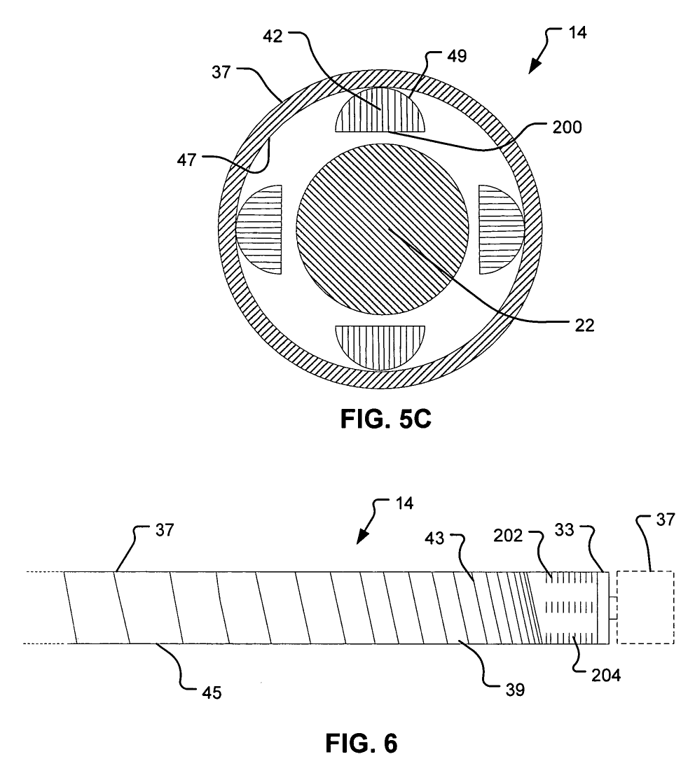

Similar to the embodiment of FIG. 3 and 3A, the guidewire 14 shown in FIG. 4 may comprise one or more reinforcing or pull wires 42. The pull wires 42 may comprise a plurality of different shapes, including, but not limited to, a rectangular wire, a flat wire, a crescent shape, a D-shape, an oval shape, or the like. As shown in FIGS. 5A to 5C, because there is no inner support tube 38 to separate the pull wire(s) 42 from the drive shaft 22, the pull wire(s) 42 may be in direct contact with the drive shaft 22. Applicants have found that rotation of the drive shaft 22 may cause twisting in the pull wires, which increases the chance of the pull wire 42 breaking. To reduce the friction between the pull wire 42 and the drive shaft 22, the pull wire 42 and/or the drive shaft 22 may be coated with Teflon® so that the drive shaft is able to rotate without causing substantial twisting of the pull wire 42.

Optionally, the pull wire may also be shaped so as to better conform with an inner surface 47 of the hollow guidewire 14. Substantially conforming a surface 49 of the pull wire 42 with the inner surface 47 of the hollow guidewire 14 increases the space between the rotating drive shaft 22 and the pull wire(s) 42 by allowing the pull wire 42 to be moved radially outward away from the drive shaft 22 and to contact the inner surface 47 at a tangential point. As shown in FIG. 5B, the surface 49 may be curved so as to conform to the curved inner surface 47 of the hypotube 37. The radius of curvature of the pull wire will typically be less than or equal to the radius of curvature of the inner surface 47 of hollow guidewire 14 so as to provide only one point of contact between the hollow guidewire and the pull wire 42.

The additional space between the drive shaft and the pull wire reduces the contact between the drive shaft 22 and the pull wire 42 and further reduces the possibility of breaking of the pull wire 42. For example, as shown in FIGS. 5A and 5B, for pull wires 42 that have substantially the same thickness T and width W, the pull wire with a surface 49 that conforms to the inner surface 47 (FIG. 5B) provides greater clearance between the drive shaft 22 and the pull wire 42 than a flat or rectangular pull wire. Additionally, the D-shaped pull wire will typically contact the inner surface 47 at one point, which reduces the friction between the pull wire and the guidewire body.

Optionally, pull wire 42 may have a flattened surface 200 adjacent the drive shaft 22. Applicants have found that having a flat surface facing the rotating drive shaft further reduces the binding and friction between the pull wire 42 and the drive shaft 22 because the rotating drive shaft would only contact the pull wire at a tangential point, therefore minimizing friction and a possibility of twisting between the pull wire and drive shaft. In alternative embodiments, however, surface 200 may be curved, if desired, but as noted, such embodiments tend to have an increased chance of tangling.

The pull wire 42 will generally have a thickness T of between about 0.002 inches and about 0.040 inches and width W between about 0.002 inches and 0.080 inches. As can be appreciated, the dimensions of pull wire 42 will depend on the dimension of the inner lumen and the largest radial dimension of the hollow guidewire 14, and the only requirement is that the pull wire fit within the inner lumen of the hollow guidewire.

When the pull wire is moved proximally, the distal tip will deflect. To improve the deflection of the distal tip of the hollow guidewire, the hypotube may optionally comprise one or more set of circumferential openings or thinned portions 202 and support ribs 204 on the distal portion of the hypotube 37, distal of the helical windings 43. If the hollow guidewire only comprises ones pull wire 42, the hollow guidewire 14 will typically only comprise one set of support ribs 204 and circumferential openings or thinned portions 202 (FIG. 4). But if the hollow guidewire comprises a plurality of pull wires 42 (FIG. 5C) the hollow guidewire 14 may comprise a corresponding number of sets of support ribs 204 and openings or thinned portions 202 (FIG. 6).

The radial slots, openings, and/or thinned portions 202 may be formed on the hypotube through laser edging that removes at least a portion of the material from the hypotube. The openings 202 will extend around less than the entire circumference of the hypotube, but if the laser merely creates thinner regions, it may be possible to have the thinner region extend completely around the hypotube. In preferred embodiments, however, the thinner portions and openings 202 typically extend between about 25% of the guidewire body (e.g., 90 degrees) and about 75% (e.g., 270 degrees) of the guidewire body.

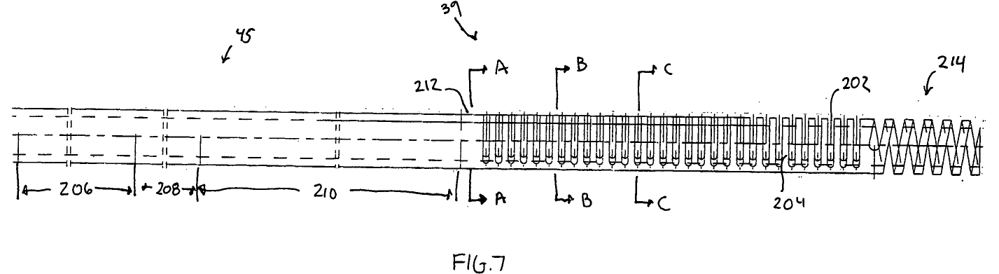

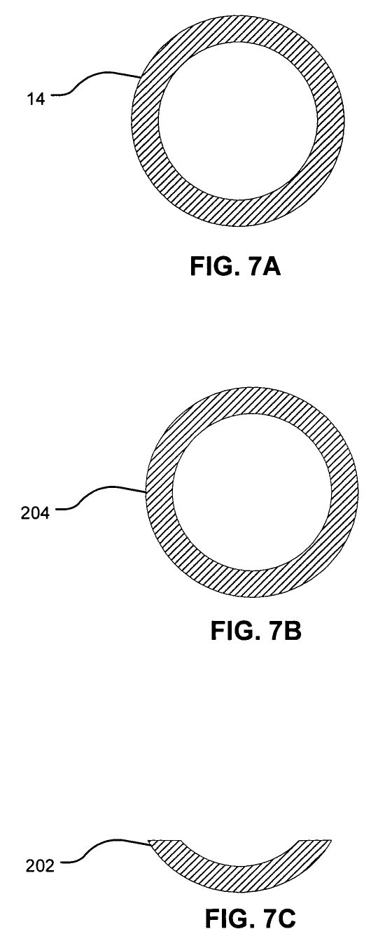

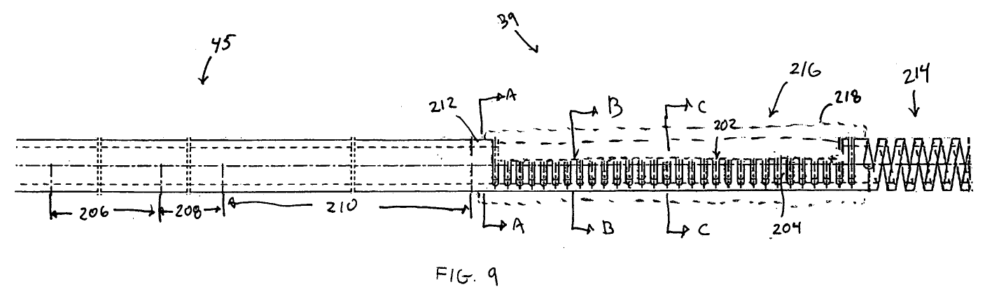

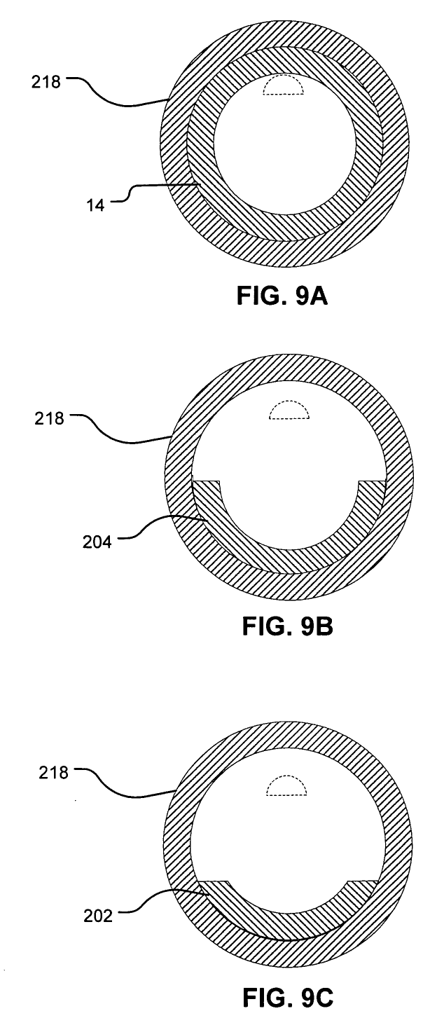

FIGS. 7 and 9 (not to scale) illustrate two additional hollow guidewire bodies 14 that encompass some of the novel aspects of the present invention. In the illustrated embodiments, a proximal portion 45 of the hollow guidewire 14 comprises one or more sections of constant pitch helical windings. Each of the sections 206, 208 vary to some degree from an adjacent section—e.g., either a different pitch from the adjacent section or one section has a left handed pitch and the other section has a right handed pitch. The sections may have the same number of helical windings or different number of helical windings. In one configuration, the hollow guidewire body comprises a first section 206 that spans 0.600 inches and has fifteen helical windings that have a pitch of 0.040 inches. The second section 208 spans 1.380 inches and has sixty-nine helical windings that have a pitch of 0.020 inches between the windings.

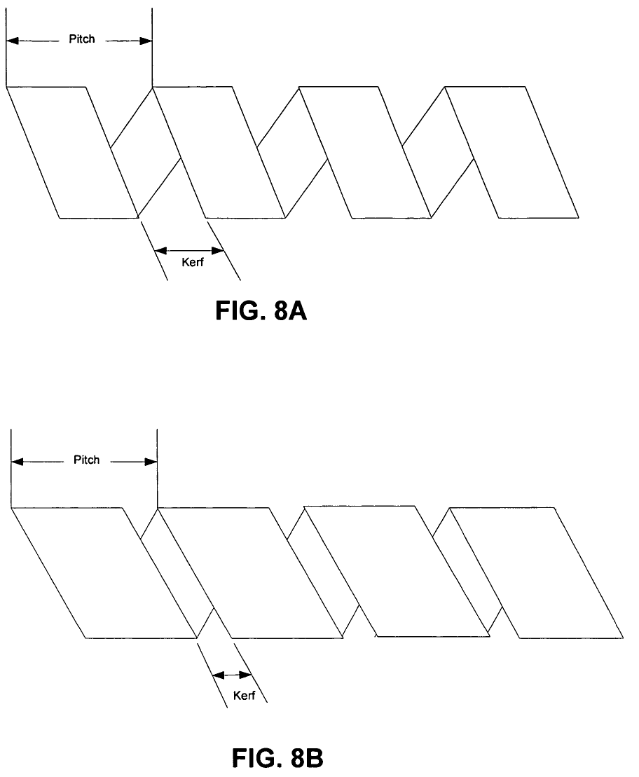

The adjacent helical windings is separated by a kerf. As shown in FIGS. 8A and 8B, the kerf typically corresponds to a width of the laser beam used to create the cuts. Applicants have found that a smaller kerf (FIG. 8B) provides improved floppiness/flexibility and torqueability of the hollow guidewire. The kerf on the hollow guidewire body 14 of the present invention typically ranges from 0.0005″- 0.004″ preferably between about 0.001″ and about 0.002,″ but may be larger or smaller as desired.

Optionally, as noted above, the hollow guidewire body 14 may also comprises a section third section 210 that is distal to sections 206, 208 that comprises a pitch that decreases in the distal direction (or increases in the distal direction). The taper may be liner or non-linear. In one configuration, the variable pitch section 210 spans 7.872 inches and has 598 variable pitches in which the proximal pitch of the section is 0.020328 inches and the distal most pitch is 0.006 inches. As can be appreciated, the hollow guidewire body 14 may comprise any number sections, and the sections may have any desired taper to the pitch.

The hollow guidewire body typically has one or more sections 212 that do not have any coils formed thereon (e.g., solid walled throughout). Typically, the sections that do not have any coils formed thereon 212 are transition areas between adjacent sections 206, 208, 210. Such transition areas 212 typically have a length between about 0.001 inches and 0.007 inches, but could be larger or smaller, if desired.

For any of the embodiments described herein, the helical coils of the hollow guidewire body 14 may be “left-handed” or “right-handed”. In some preferred embodiments, however, the different sections 206, 208, 210 of helical coils will have at least one left-handed coil section and at least one right-handed coil section. Typically, the left handed coil sections and the right handed coil sections are alternating along a length of the hollow guidewire body 141. As can be appreciated, when a right handed torque is applied to a coil that comprises all right-handed coils, the coils will torque without substantial “opening” of the coils. However, if a left-handed torque is applied to the same right-handed coils, the coils will tend to open and may affect the 1:1 torque transmission through the guidewire body 14. While the smaller kerf has been found to improve torque transmission, Applicants have found that having at least one left-handed section and at least one right-handed section further compensates for the opening of the coils when a torquing force is applied to the proximal end of the guidewire body. Consequently, similar amounts of torque may be transmitted to a distal tip of the hollow guidewire body when applying either a left-handed or right-handed torque.

Optionally, the hollow guidewire may comprise an integrally formed coil 214 at the distal tip. The distal coil 214 may be configured to threadedly receive a radiopaque coil (not shown), such as a platinum coil. The radiopaque coil may be soldered, glued, or otherwise attached to the distal coil 214 so as to provide a radiopaque marker for fluoroscopic tracking of the hollow guidewire body 14. The distal coil 214 may have any desired length and pitch, but in one exemplary configuration, the distal coil 214 is 0.027 inches long and has 5.75 helical windings that have a kerf of 0.0028 inches and a pitch of 0.005 inches.

Similar to the embodiments illustrated in FIGS. 4 and 6, the embodiments of FIGS. 7 and 9 may comprise a plurality of openings 202 and support ribs 204 to improve the bendability/deflectability of the distal portion of the guidewire body 14. A support rib 204 will typically be disposed between each opening 202. The openings 202 may take on a variety of different forms and may extend over any desired length of the distal portion. Each rib 204 along the distal portion may have a constant thickness in the axial direction or the ribs 204 may have a variable thickness along the axial length of the hollow guidewire body 14 (e.g., an axial thickness of a proximal most rib may be thicker or thinner than an axial thickness of a distal most rib). Moreover, each rib may extend completely around a circumference of the hollow guidewire body 14 or only around a portion of the hollow guidewire body. As shown in FIGS. 7A to 7C and 9A to 9C, the support ribs 204 typically will extend between 100% (e.g., 360 degrees) and about 25% (e.g., 90 degrees) around the circumference of the hollow guidewire body 14. The thinned portions 202 (FIGS. 7C and 9C) will typically extend between about 25% (90 degrees) and about 75% (e.g., 270 degrees) of the hollow guidewire body 14.

For the embodiments of FIG. 9, if the ribs 204 extend around less than 100% of the circumference of the hollow guidewire, the pull wire (not shown) may be exposed through A window 216 created by the ribs 204 and openings 202. In such embodiments, a flexible tubing 218 may be placed over the ribs 204 and openings 202 so as to protect the pull wire (shown in dotted lines in FIGS. 9A to 9C). The flexible material may be comprised of a polymeric material, including, but not limited to polyethylene, Teflon®, or the like.

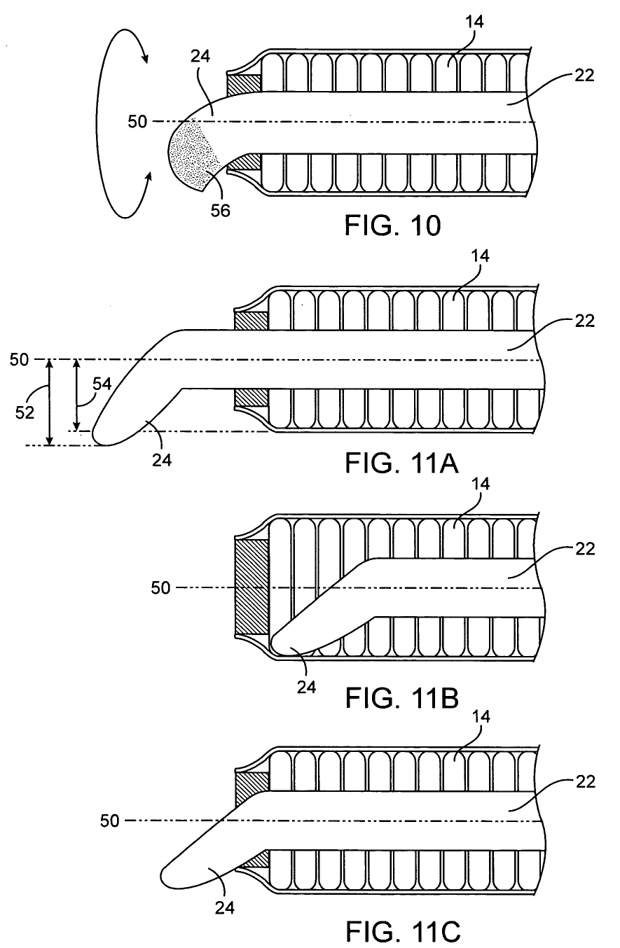

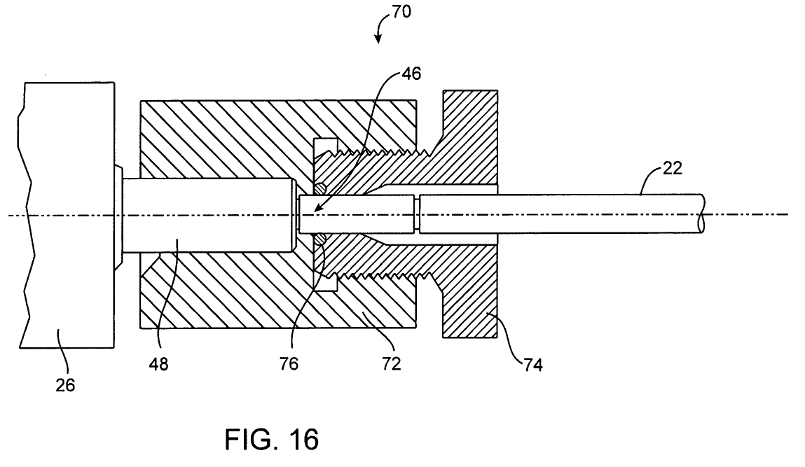

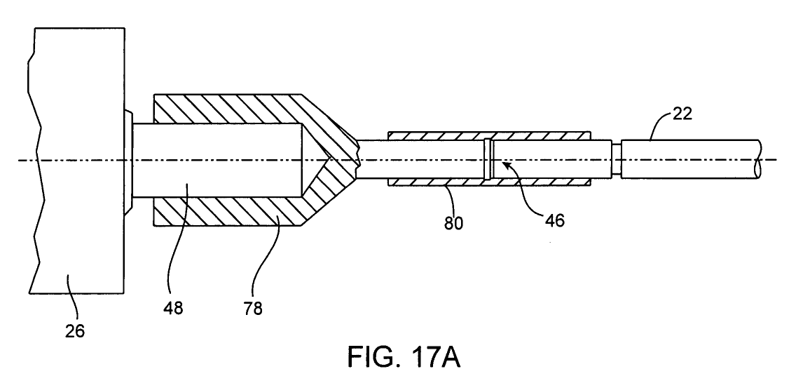

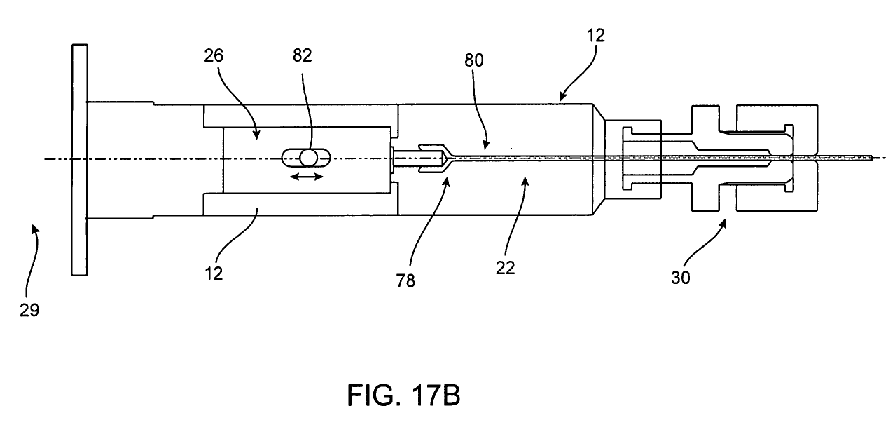

FIGS. 10-15 show various embodiments of the drive shaft 22 of the present invention. In most embodiments, the drive shaft 22 is a wire, a counter-wound multiple strand wire, or a plurality of braided wires having a body and a shaped distal tip 24. The proximal end of the drive shaft 22 can be removably coupled to a rotatable motor shaft 48 (FIGS. 16 and 17A) or manually manipulated (FIG. 2). The body of the drive shaft 22 extends through the elongate member 14 so that the distal tip 24 of the drive shaft is positioned near the distal end of the elongate member 14. The detachable connection to the motor shaft 48 allows the drive shaft 22 and elongate member 14 to be detached from the motor shaft 48 and connector assembly 30 so that an access or support system can be placed over the elongate member 14 and advanced through the body lumen.

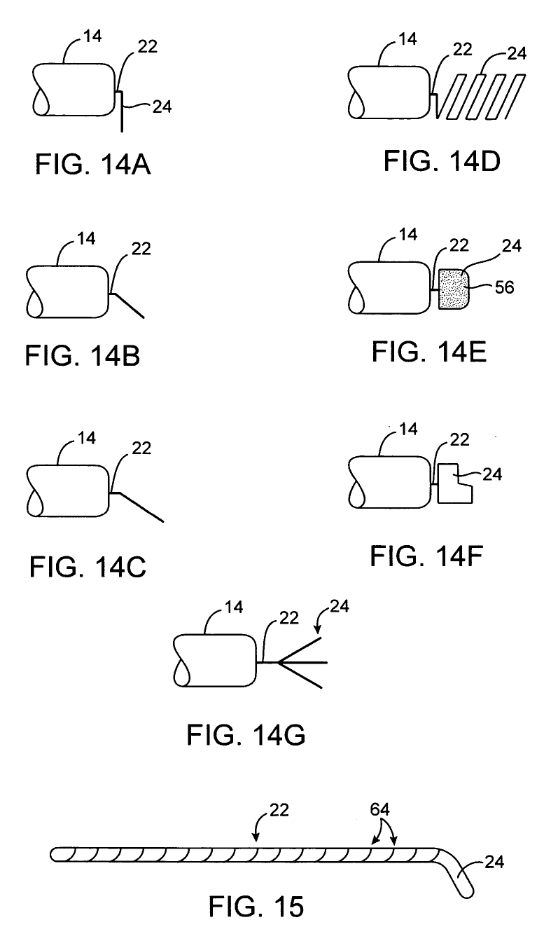

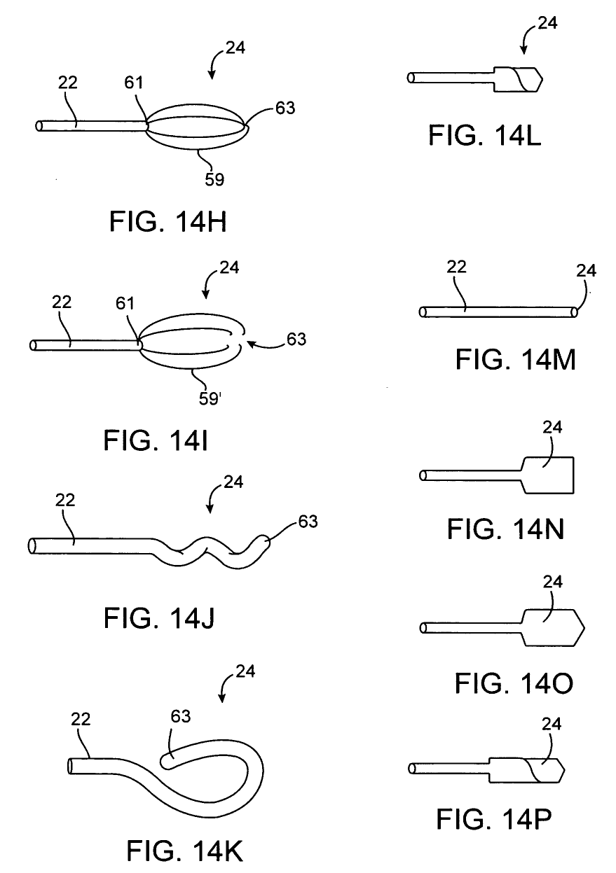

As shown in FIG. 10 and 11A-11C, the distal tip can be shaped or deflected from the longitudinal axis 50 to extend beyond the radius of the elongate member 14 such that rotation of the drive shaft 22 creates a path radius 52 that is as at least as large as the radius 54 of the distal end of the elongate member 14. In other embodiments, the distal tip 24 will be deflected and shaped so as to create a path radius 52 which is the same or smaller than the radius of the distal end of the elongate member 14 (FIGS. 14B-14G). For example, in one exemplary configuration shown in FIG. 11C, a portion of the distal tip 24 extends beyond the distal end 18 of the elongate member when in the fully retracted position. When the drive shaft 22 is advanced out of the elongate member 14, the flexible distal tip 24 maintains a deflected shape (FIG. 11A). In alternative configurations, it is contemplated that the deflection at the distal tip 24 can straighten somewhat under the force from the walls of the elongate member 14 when the drive shaft 22 is retracted into the elongate member 14 (FIG. 11B). Thus, in the axially retracted configuration, the drive shaft 22 will have a profile that is smaller than the radius of the distal tip of the elongate member. When the drive shaft is advanced out of the distal end of the elongate member, the drive shaft will expand to an axially extended configuration in which the distal tip of the drive shaft 22 will have a profile that is larger than the axially retracted configuration, and in some embodiments will have a larger profile than the distal end of the elongate member 14.

Referring again to FIG. 10, in some configurations a layer of abrasive material 56 can be attached and distributed over at least a portion of the distal tip 24 of the drive shaft 22 so that the abrasive material 56 engages the stenotic or occlusive material as the drive shaft 22 is advanced into the occlusion or stenosis. The abrasive material 56 can be diamond powder, diamond chips, fused silica, titanium nitride, tungsten carbide, aluminum oxide, boron carbide, or other conventional abrasive particles.

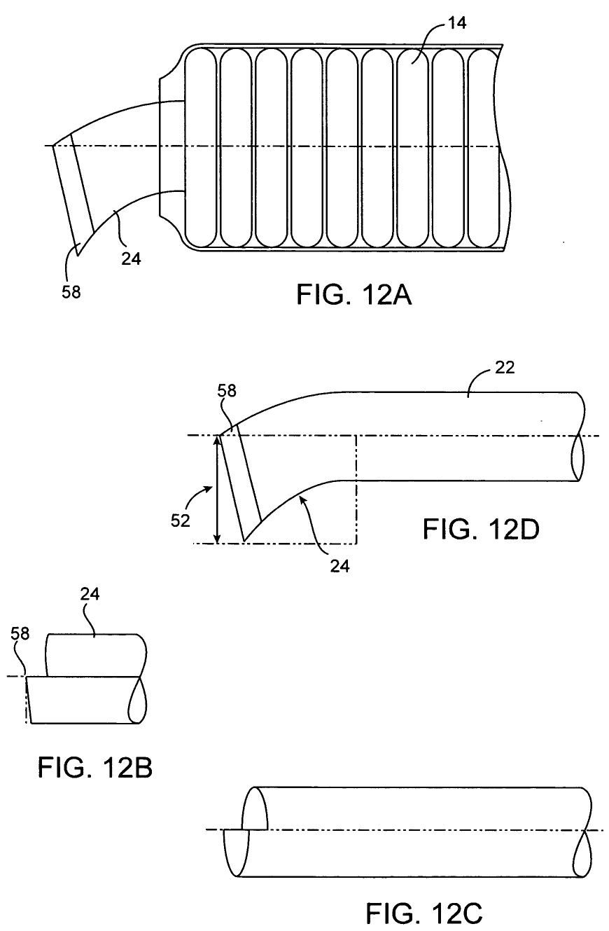

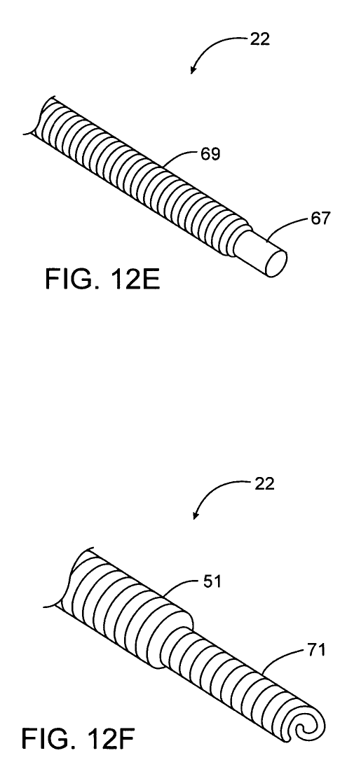

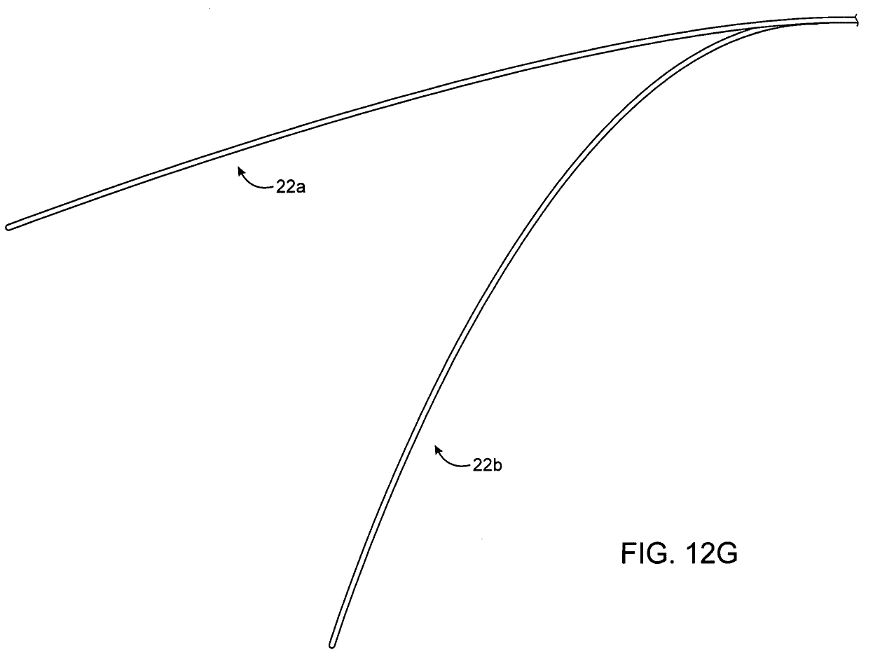

Alternatively, as shown in FIGS. 12A-12D, the distal tip 24 of the drive shaft 22 can be sharpened to facilitate passing through the occlusion or stenosis. A distal edge of the tip 24 can be sharpened so as to define a cutting edge 58 which rotatably contacts the occlusive or stenotic material. In an exemplary embodiment illustrated in FIGS. 12B-12C, a tip of the drive shaft can be sharpened to create a plurality of cutting edges 58. Furthermore, as shown in FIG. 12D and as described above, the distal tip 24 can be deflected from its longitudinal axis 50 to create the cutting path radius 52 of the drive shaft 24 that is smaller, larger, or the same length as the radius of the elongate member 14.

The drive shaft 22 can be composed of a shape retaining material, a rigid material, a flexible material, or can be composed of a plurality of materials. For example in some configurations, the drive shaft 22 can be comprised of nitinol, stainless steel, platinum-iridium, or the like. The distal tip 24 of the drive shaft 22 can have an enlarged tip, a preformed curve, or a preformed deflection (FIG. 11A). FIGS. 12E and 12F show exemplary embodiments of a counter-wound and composite drive shafts of the present invention. The counter-wound drive shaft 22 shown in FIG. 12E is made of a 0.004 inch OD center wire 67 having a right-hand wound surrounding wire 69 coiled around the center wire 67. The surrounding wire 69 can be soldered to the center wire at both ends of the center wire. In the embodiment of FIG. 12F, multiple strand wires 51 can be wound around a central coil 71 to form the drive shaft 22. The counter-wound drive shafts are significantly more flexible than a single wire guidewire and allows for a tighter bending radius over conventional guidewire. FIG. 12G illustrates the flexibility of both a 0.007 inch OD single wire stainless steel wire drive shaft 22a and a 0.007 inch OD counter-wound stainless steel drive shaft 22b. As shown by FIG. 12G, the counter-wound drive shaft has better flexibility, while still maintaining its torqueability, maneuverability, and columnar strength.

Additionally, in some embodiments, the distal portion of the drive shaft 22 is radiopaque so that a physician can track the position of the drive shaft 22 using fluoroscopy. The drive shaft 24 typically has a diameter between approximately 0.010 inches and 0.005 inches. It should be appreciated that the dimension of the drive shaft will be slightly less than the inner diameter of the hollow guidewire so as to allow rotation without significant heat generation. Consequently, the dimensions of the drive shaft will vary depending on the relative inner diameter of the elongate member 14 and the present invention is not limited to the above described dimensions of the drive shaft.

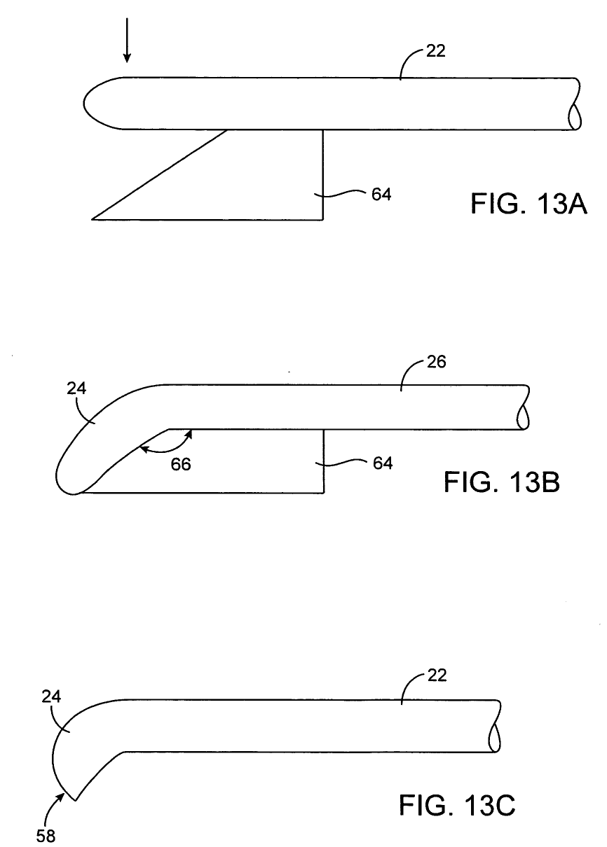

In one embodiment, the distal tip 24 of the drive shaft is created using a shaped fixture 64. As shown in FIGS. 13A and 13B, the distal tip 24 is positioned on the fixture 64 and bent to a desired angle 66. The distal tip 24 can be bent to almost any angle 66 between 0° degrees and 90° degrees from the longitudinal axis 50, but is preferably deflected between 0° degrees and 50° degrees. As shown in FIG. 13C, a sharpened edge 58 can be created on the distal tip using a wafer dicing machine used in the production of silicon microchips (not shown). The angle of the sharpened edge 58 can be almost any angle, but the angle is typically between 0° degrees and 45° degrees, and is preferably between approximately 8° degrees and 18° degrees. Naturally, it will be appreciated that a variety of methods can be used to manufacture the distal tip of the drive shaft and that the present invention is not limited to drive shafts produced by the described method.

As mentioned above, the distal tip 24 can take various shapes. One embodiment having a deflected distal tip 24 is shown in FIG. 14A. In an exemplary configuration, the deflected tip is offset at an angle such that rotation of the drive wire 22 defines a profile or path that is at least as large as the outer diameter of the distal end of the elongate member 14. As shown in FIGS. 14B and 14C, in other embodiments, the tip can be deflected at other angles and may have a length that creates a path that is smaller or the same diameter as the distal end of the elongate member. The deflected distal tip can extend radially any feasible length beyond the perimeter or diameter of the elongate member 14. It should be understood that the invention is not limited to a single deflected tip. For example, the drive shaft can comprise a plurality of deflected tips. Alternatively, the drive shaft may have a distal tip 24 that is twizzle shaped, spring shaped, twisted metal shaped (FIG. 14D), ball shaped (FIG. 14E), a discontinuous surface (FIG. 14F), or the like. Alternatively, the drive shaft may comprise a plurality of filaments (FIG. 14G), rigid or flexible brush elements, a plurality of coils, or the like.

The distal tip of the drive shaft can be configured optimally for the type of occlusion or stenosis to be penetrated. Some lesions are made up substantially of clot or thrombotic material that is soft and gelatinous. FIGS. 14H and 14K shows distal tip embodiments which may be used to macerate a soft clot, thrombotic material, or stenosis. FIG. 14H shows a distal tip 24 having a basket like construction which is made up of a plurality of strands 59 that are connected at their ends 61, 63. In another embodiment illustrated in FIG. 141, the distal tip 24 can be composed of a plurality of strands 59 that are unconnected at their distal ends 63. Additionally, the distal ends 63 of the strands 59 can be turned inward so that the distal ends 63 do not penetrate the body lumen when rotated. FIG. 14J shows a corkscrew spiral distal tip having a blunt distal end 63. FIG. 14K shows a distal tip having a loop configuration.

In another exemplary embodiment shown in FIG. 14L, the distal tip 24 of the drive shaft 22 can be flattened and twisted to create a screw like tip that can create a path through the occlusion. The flattened and twisted distal tip 24 can have a same width, a smaller width or a larger width than the drive shaft 24. For example, in one configuration for a drive shaft having an outer diameter of 0.007 inches, the distal tip 24 can be flattened to have a width between approximately 0.015 inches and 0.016 inches, or more. It should be appreciated, however, that the distal tip can be manufactured to a variety of sizes.

FIGS. 14M-814P show one method of manufacturing the flattened and distal tip of the present invention. The round drive shaft 22 (FIG. 14M) is taken and the distal end is flattened (FIG. 14N). The distal end can be sharpened (FIG. 14O) and twisted two or two and a half turns (FIG. 14P). If a different amount of twists are desired, the distal tip can be manufactured to create more (or less) turns.

In use, the distal tip 24 is rotated and advanced distally from a retracted position to an extended position into the soft material in the target lesion. If slow speed rotation is desired the user can rotate the drive shaft slowly by hand by grasping a knurled knob attached to the proximal end of the drive shaft (FIG. 2). If high speed rotation is desired, the proximal end of the drive shaft 22 can be attached to the drive motor 26. As the expanded wire basket tip is rotated, the tip macerates the soft clot and separates the clot from the wall of the body lumen. If a large diameter hollow guidewire working channel is used to deliver the drive shaft to the target area, the macerated clot can be aspirated through the guidewire working channel. Alternatively or additionally, a fluid, such as thrombolytic agents, can be delivered through the working channel to dissolve the clot to prevent “distal trash” and blockage of the vasculature with debris from the macerated clot.

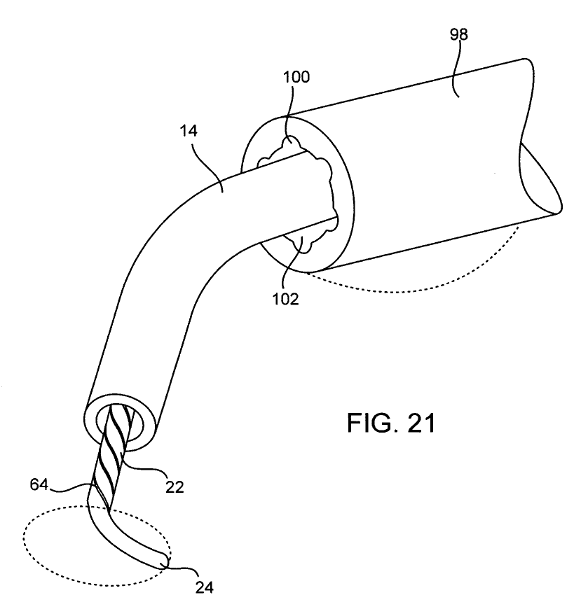

As shown in FIGS. 15 and 21 in some embodiments the drive shaft 22 can optionally have spiral threads or external riflings 64 which extend along the body 44. As the drive shaft 22 is rotated and axially advanced into the atheromatous material, the distal tip 24 creates a path and removes the atheromatous material from the blood vessel. The rotating spirals 64 act similar to an “Archimedes Screw” and transport the removed material proximally through the axial lumen of the elongate member 14 and prevent the loose atheromatous material from blocking the axial lumen of the elongate member 14 or from escaping into the blood stream.

In use, drive shaft 24 is rotated and advanced to create a path distal of the elongate member 14 to create a path through the occlusion. The drive shaft 24 can be advanced and rotated simultaneously, rotated first and then advanced, or advanced first and then rotated. The drive shaft 22 is typically ramped up from a static position (i.e. 0 rpm) to about 5,000 rpm, 20,000 rpm with a motor. It should be noted, however, that the speed of rotation can be varied (higher or lower) depending on the capacity of the motor, the dimensions of the drive shaft and the elongate member, the type of occlusion to be bypassed, and the like. For example, if desired, the drive shaft can be manually rotated or reciprocated at a lower speed to macerate soft clots or to pass through lesions.

The distal tip of the drive shaft 22 can extend almost any length beyond the distal portion of the hollow guidewire. In most embodiments, however, the distal tip typically extends about 5 centimeters, more preferably from 0.05 centimeters to 5 centimeters, and most preferably between 0.05 centimeter and 2 centimeters beyond the distal portion of the hollow guidewire.

Referring now to FIGS. 16, 17A, and 17B, the motor shaft 48 and the proximal end 46 of the drive shaft 22 are coupled together with a detachable linkage assembly 70. In one embodiment shown in FIG. 16, linkage assembly 70 has a first flange 72 attached to the motor shaft 48. The first flange can be snap fit, snug fit, or permanently attached to the drive shaft 48. A second flange 74 can be permanently or removably coupled to the proximal end 46 of the drive shaft 22 so that the first flange 72 of the motor shaft 48 can threadedly engage the second flange 74. In some embodiments, the proximal end of the drive shaft 46 can be enlarged so as to improve the engagement with the second flange 74. An o-ring 76 is preferably disposed within a cavity in the first flange 72 to hold the first flange 72 and second flange 74 in fixed position relative to each other.

As shown generally in FIGS. 1 and 17B, the motor 26 can be removably coupled to the housing 12. To detach the motor 26 and power supply 28 from the drive shaft 22, the user can unlock the luer assembly 30 so as to release the elongate member 14 from the housing 12. The drive shaft 22 and elongate member 14 are then both free to move axially. The motor 26 can be moved proximally out of the housing 12 and the proximal end 46 of the drive shaft 22 can be detached from the motor shaft 48. After the motor 26, housing 12, and luer assembly 30 have been uncoupled from the elongate member 14 and drive shaft 22, a support or access system (not shown) can be advanced over the free proximal end of the elongate member 14. Thereafter, the luer assembly and motor shaft 48 can be recoupled to the elongate member 14.

In the embodiment shown in FIGS. 17A and 17B, the-linkage assembly 70 includes a connecting shaft 78 that can be snugly fit over the motor shaft 48. The connecting shaft 78 preferably tapers from a diameter slightly larger than the motor shaft 48 to a diameter of that of the approximately the proximal end 46 of the drive shaft 22. In the embodiment shown, the connecting shaft 78 is coupled to the drive shaft through shrinkable tubing 80. Because the connecting shaft 78 is snug fit over the motor shaft, (and is not threadedly attached to the drive shaft) the size of the connecting shaft 78 can be smaller than the linkage assembly 70. While the exemplary embodiments of the connection assembly between the drive shaft and motor shaft have been described, it will be appreciated that drive shaft and motor shaft can be attached through any other conventional means. For example, the motor shaft 48 can be coupled to the drive shaft 22 through adhesive, welding, a snap fit assembly, or the like.

As shown in FIG. 17B, the drive shaft 22 extends proximally through the housing 12 and is coupled to the motor shaft 48. An actuator 82 can be activated to advance and retract the drive shaft 22. In some embodiments, the motor is press fit into the actuator housing 12. The drive shaft 22 is attached to the motor shaft 26 via o-rings such that the drive shaft 22 can be moved axially through axial movement of the actuator 82.

In most embodiments, actuation of the drive motor 26 and power supply 28 (e.g. rotation of the drive shaft) will be controlled independent from advancement of the drive shaft 22. However, while the actuator 82 is shown separate from the control system 27 and power supply 28 (FIG. 1), it will be appreciated that actuator 82 and control system 27 can be part of a single, consolidated console attached to the housing 12 or separate from the housing 12. For example, it is contemplated that that the drive shaft 22 can be rotated and advanced simultaneously by activation of a single actuator (not shown).

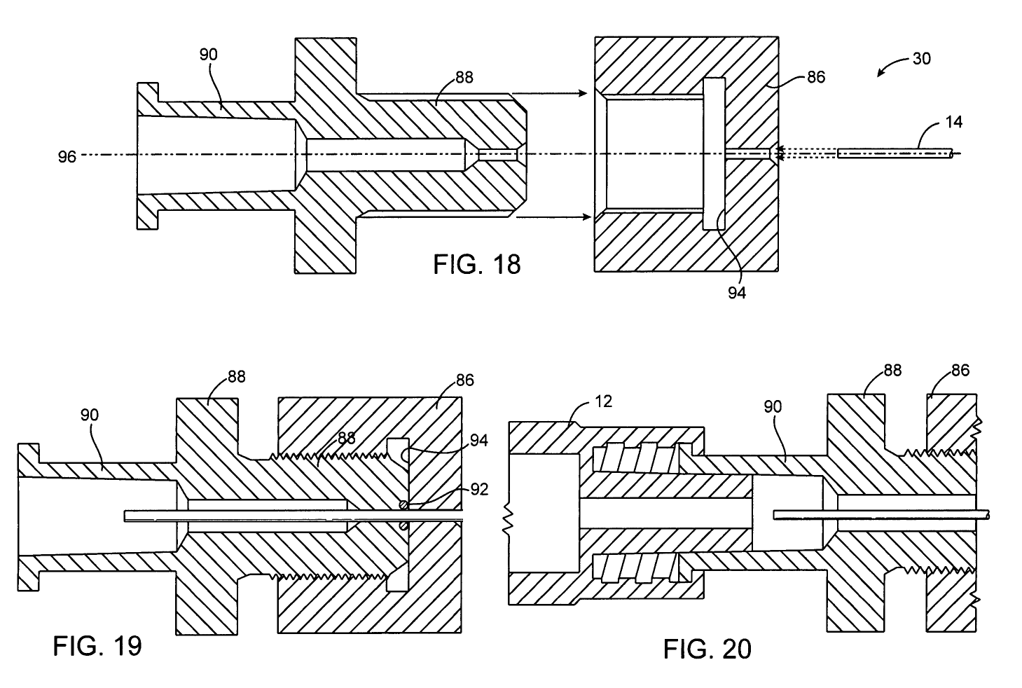

A connection assembly 30 is positioned on a proximal end of the housing to couple the elongate member 14 and the drive shaft 22 to the housing 12. In a preferred embodiment shown in FIGS. 18-20, the connection assembly 30 is a detachable luer which allows the drive shaft 22 to be moved (e.g. rotated, reciprocated, translated) while the elongate member is maintained in a substantially static position. FIG. 18 best illustrates an exemplary luer connection assembly 30 which couples the elongate member 14 and the housing 12. The luer has a gland 86 which is rotatably connected to a fitting 88 and a tubular portion 90. Rotation of the gland 86 rotates and torques the elongate member 14 while the elongate member 14 is advanced through the blood vessel. Fitting 88 is threaded into the gland 86 such that a distal end of the fitting engages an o-ring 92 and a surface wall 94 of the gland. The longitudinal axis 96 of the fitting 88 and gland 86 are aligned so as to be able to receive the axial lumen of the elongate member 14. As the fitting 88 engages the o-ring 92, the o-ring is compressed radially inward to squeeze and maintain the position of the elongate member 14. Accordingly, as illustrated in FIG. 19, when the drive shaft 22 is rotated within the elongate member 14, the o-ring 92 is able to substantially maintain the position and orientation of the elongate member 14. Tubular portion 90 attached to the proximal end of the fitting 88 threadedly engages the housing 12 and enables the luer connection assembly 30 to be removed from the housing 12 (FIG. 20). A more complete description of the connection assembly 30 can be found in commonly owned U.S. patent application Ser. No. 09/030,657, filed Feb. 25, 1998, the complete disclosure of which was previously incorporated by reference. It should be appreciated that the present invention is not limited to the specific luer assembly described. Any luer assembly can be used to connect the elongate member 14 to the housing 12. For example, a Y-luer assembly (not shown) can be used with the system of the present invention to infuse or aspirate of fluids through the lumen of the hollow guidewire 14.

As shown in FIG. 21, systems of the present invention can further include an access or support system 98. The access or support system 98 can be an intravascular catheter such as a hollow guidewire support device, support catheter, balloon dilation catheter, atherectomy catheters, rotational catheters, extractional catheters, conventional guiding catheters, an ultrasound catheter, a stenting catheter, or the like. In an exemplary configuration shown in FIG. 21, the system includes an infusion or aspiration catheter which has at least one axial channel 100, and preferably a plurality of axial channels 100 which extends through the catheter lumen 102 to the distal end of the catheter. The elongate member 14 and drive shaft 22 can be positioned and advanced through the lumen 102 of the catheter. The axial channel 20 of the elongate member 14 and/or the axial channels 100 of the catheter 98 can also be used to aspirate the target site or infuse therapeutic, diagnostic material, rinsing materials, dyes, or the like.

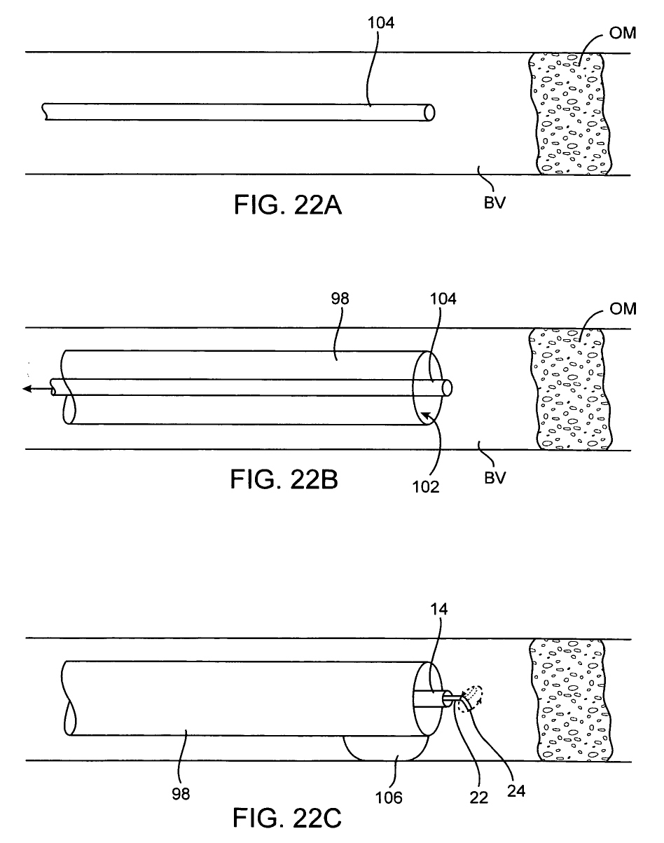

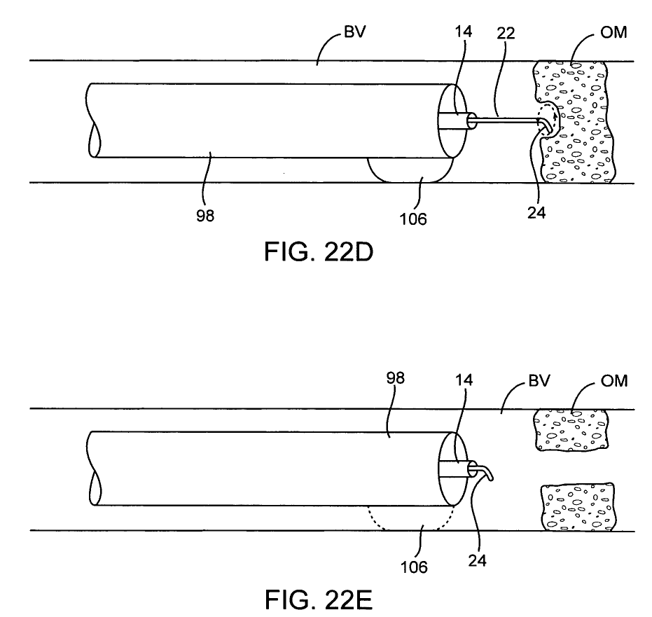

The access or support system can be guided by the elongate member to the target site in a variety of ways. For example, as illustrated in FIGS. 22A to 22E, a conventional guidewire 104 can be advanced through the blood vessel BV from the access site (FIG. 22A). Once the guidewire 104 has reached the target site, the support or access system 98 can be advanced over the guidewire 104 (FIG. 22B). Alternatively, the guidewire 104 and support or access system 98 can be simultaneously advanced through the body lumen (not shown). Once the support or access system 98 has reached the target site, the conventional guidewire 104 can be removed and the hollow guidewire 14 having the drive shaft 22 can be introduced through the lumen 102 of the access system 98 (FIG. 22C). Even if the distal tip 24 of the drive shaft 22 is not fully retracted into the axial lumen 20, the lumen 102 of the support or access system protects the blood vessel BV from damage from the exposed distal tip 22. In most methods, the support or access system is positioned or stabilized with balloons, wires, or other stabilization devices 106 to provide a more controlled removal of the occlusive or stenotic material OM. Once the hollow guidewire 14 and drive shaft 22 have reached the target site, the drive shaft can be rotated and advanced into the occlusive or stenotic material OM to create a path (FIGS. 22D and 22E).

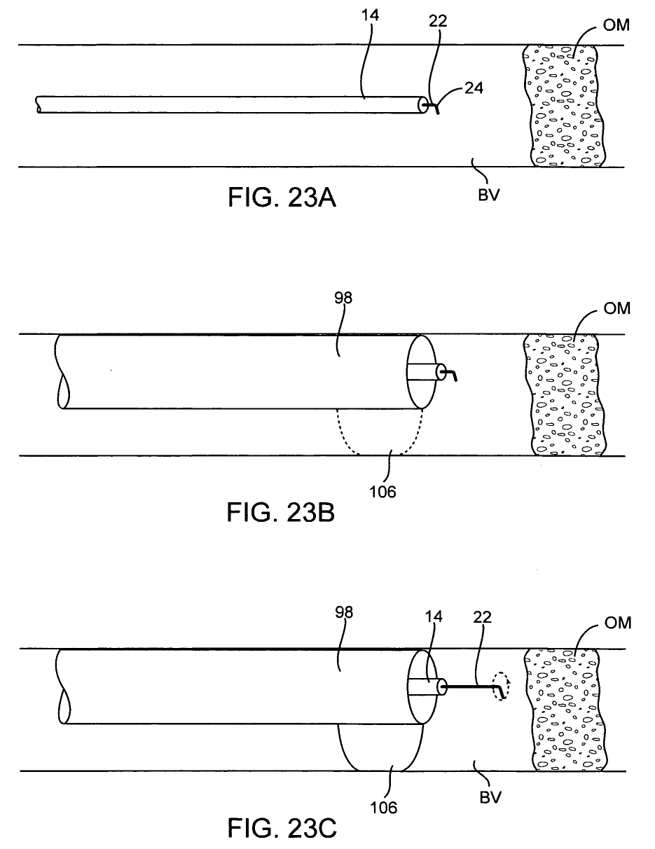

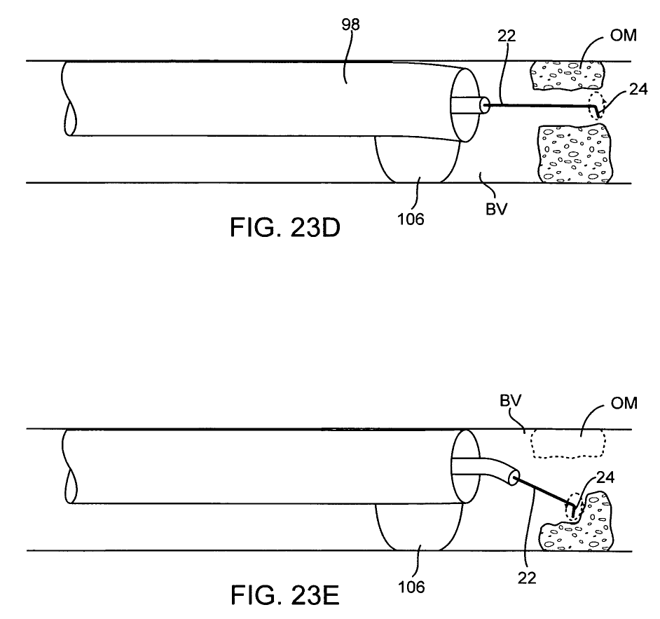

In another method of the present invention, the hollow guidewire 14 can be used to guide the support or access system to the target site without the use of a separate guide wire. The hollow guidewire 14 provides the flexibility, maneuverability, torqueability (usually 1:1), and columnar strength necessary for accurately advancing through the tortuous vasculature and positioning the distal end of the support or access system at the target site. The steerable distal portion can be deflected and steered through the tortuous regions of the vasculature to get to the target site. As shown in FIG. 23A, the hollow guidewire is advanced through the tortuous blood vessel to the target site. Due to the small size of the guidewire 14 relative to the blood vessel, even if the distal tip 24 of the drive shaft 22 extends partially out of the hollow guidewire 14, any potential damage to the blood vessel BV will be minimal.

Once the hollow guidewire reaches the target site within the blood vessel, the motor shaft 48, luer assembly 30, and housing 12 can be detached from the proximal end 46 of the drive shaft 22 so that the support or access system can be placed over the hollow guidewire. After the motor has been detached, the support or access system can be advanced over the guidewire and through the body lumen to the target site (FIG. 23B). To reattach the drive motor 26 to the drive shaft 22, the hollow guidewire 14 and drive shaft 22 are inserted through the luer assembly 30. The luer assembly 30 is tightened to lock the position of the hollow guidewire 14. The drive shaft 22 will extend proximally through the housing 12 where it can be recoupled to the motor shaft using the above described linkage assemblies 70 or other conventional linkage assemblies. Once at the target site, the position of the support or access system 98 can be stabilized by a balloon, wires, or other stabilizing devices 106, and the drive shaft 22 can be rotated and advanced into the occlusive or stenotic material OM (FIGS. 23C and 23D). The rotation of the drive shaft creates a path forward of the distal end 18 of the hollow guidewire 14. As noted above, the path can have the same diameter, smaller diameter, or larger diameter than the distal end of the hollow guidewire. Before, during, or after the rotation of the drive shaft, the user can steer or deflect the distal end 18 of the hollow guidewire 14 to guide the hollow guidewire to the desired location within the blood vessel. For example, as shown in FIG. 23E, once a portion of the occlusion or stenosis has been removed, the distal end 18 of the hollow guidewire 14 can be guided to angle the distal end so that the drive shaft is extended into a different portion of the occlusive or stenotic material OM.

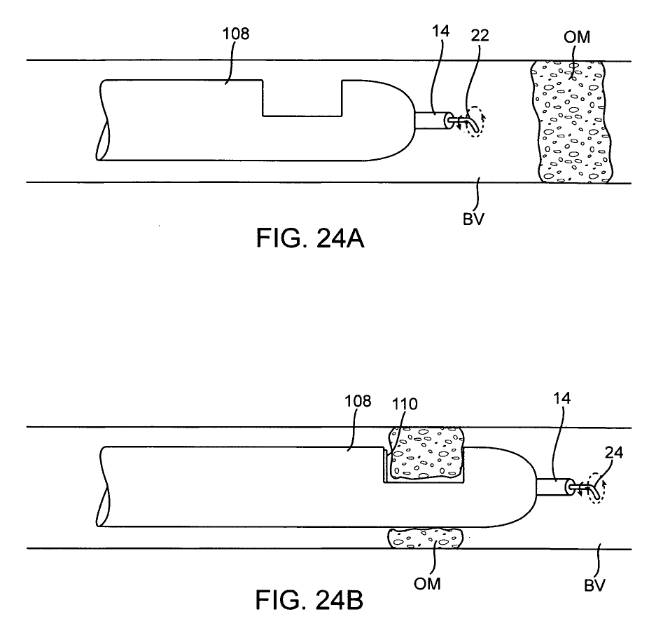

While the apparatus of the present invention is sufficient to create a path through the occlusion OM without the use of a support or access system, the apparatus 10 of the present invention can be used in conjunction with other atherectomy devices to facilitate improved removal or enlargement of the path through the occlusion. For example as shown in the above figures, the hollow guidewire 14 and the atherectomy device 108 can be advanced through the body lumen and positioned adjacent the occlusion OM. The drive shaft 22 is rotated and advanced to make an initial path through the occlusion (FIG. 24A). The hollow guidewire 14 is then moved through the path in the occlusion and the atherectomy device 108 can then be advanced over the hollow guidewire 14 into the path in the occlusion OM to remove the remaining occlusion with cutting blades 110, or the like (FIG. 24B). While FIG. 24B shows cutting blades 110 to remove the occlusive material OM, it will be appreciated that other removal devices and techniques can be used. Some examples include balloon dilation catheters, other atherectomy catheters, rotational catheters, extractional catheters, laser ablation catheters, stenting catheters, and the like.

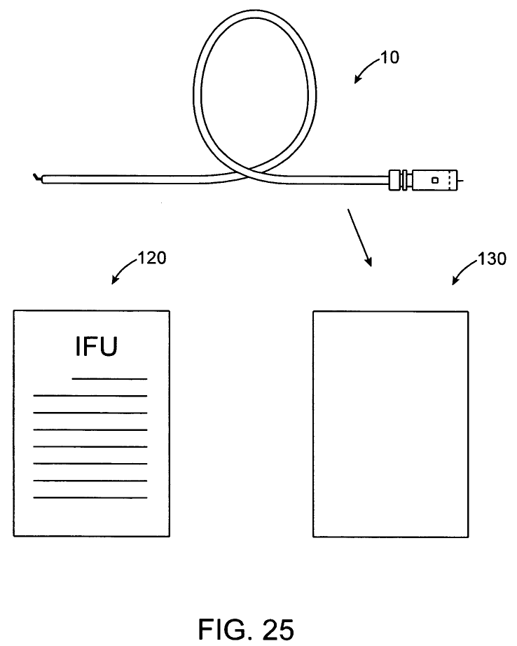

In another aspect, the invention provides medical kits. As shown in FIG. 25, the medical kit generally includes a system 10, instructions for use (IFU) 120 which describe any of the above described methods, and a package 130. The IFU can be separate from the package or they can be printed on the package. The kits can also optionally include any combination of a second guidewire, a motor, a power supply, a plastic sheath cover, connection assemblies, support or access systems, or the like.

While the above is a complete description of the preferred embodiments of the invention, various alternatives, modifications, and equivalents may be used. For example, while the above description focuses on a rotatable drive shaft to remove material from the body lumen, the hollow guidewires of the present invention may incorporate other tissue removal assemblies. The tissue removal assembles may be fixedly positioned at the distal tip of the hollow guidewire or movable between a first position (e.g., retracted position) and a second position (e.g., deployed position). The tissue removal assembly may take on the form of a laser, LED, RF electrode or other heating element, an ultrasound transducer or the like. Thus, instead of a drive shaft, the above tissue removal assemblies may have a lead extend through the axial lumen to the tissue removal assembly that is fixedly or movably positioned at or near the distal end of the hollow guidewire. Moreover, while not explicitly illustrated, a person of ordinary skill in the art will recognize that aspects of one configuration of the hollow guidewire body may be used with other configurations of the hollow guidewire body. For example, while the guidewire body of FIG. 2 does not show thinned portions 202 near the distal end or varying pitch coils on its proximal portion, such a configuration would be encompassed by the present invention. Therefore, the above description should not be taken as limiting the scope of the invention which is defined by the appended claims.

1. A steerable hollow guidewire comprising:
an elongate hollow guidewire body comprising a proximal opening, a distal opening, and an axial lumen extending from the proximal opening to the distal opening;
a rotatable drive shaft disposed within the axial lumen, wherein a distal tip of the rotatable drive shaft is adapted to extend distally through the distal opening in the guidewire body; and
at least one pull wire extending through the axial lumen and coupled to a distal end portion of the guidewire body, the pull wire(s) comprising a curved surface that substantially corresponds to a shape of an inner surface of the axial lumen.

2. The steerable hollow guidewire of claim 1 wherein the hollow guidewire body comprises a solid wall tubular proximal portion integrally formed with the distal end portion, wherein the distal end portion comprises helical windings formed thereon.

3. The steerable hollow guidewire of claim 2 wherein the guidewire body comprises a single hypotube.

4. The steerable hollow guidewire of claim 2 wherein a pitch between adjacent helical windings decreases in the distal direction so as to increase a flexibility in the distal direction along the distal portion of the guidewire body.

5. The steerable hollow guidewire of claim 1 wherein the guidewire body has a largest radial dimension between about 0.009 inches and about 0.035 inches.

6. The steerable hollow guidewire of claim 1 wherein the distal end portion of the guidewire body comprises a plurality of openings or thinned portions that extend circumferentially about at least a portion of the distal end portion of the guidewire body.

7. The steerable hollow guidewire of claim 1 comprising a radiopaque marker disposed at the distal end portion of the guidewire body.

8. The steerable hollow guidewire of claim 1 wherein the pull wire further comprises a substantially flat surface that faces the rotatable shaft.

9. The steerable hollow guidewire of claim 1 wherein the pull wire comprises a D-shaped cross-section.

10. The steerable hollow guidewire of claim 1 wherein the pull wire comprises a crescent shaped cross section or oval shaped cross section.

11. The steerable hollow guidewire of claim 1 wherein the rotatable drive shaft is rotatable and advanceable axially within the axial lumen.

12. The steerable hollow guidewire of claim 1 wherein the at least one pull wire(s) comprise a single pull wire.

13. The steerable hollow guidewire of claim 1 wherein the at least one pull wire(s) comprises two or more pull wires.

14. The steerable hollow guidewire of claim 1 wherein at least one of the pull wire(s) and the rotatable drive shaft are at least partially coated with Teflon.

15. The steerable hollow guidewire of claim 1 comprising a removable housing coupled to a proximal portion of the hollow guidewire body.

16. A hollow guidewire comprising a hypotube that comprises a proximal portion and a distal portion, wherein at least a part of the distal portion of the hypotube comprise helical windings formed thereon so that the distal portion of the hypotube is more flexible than the proximal portion.

17. The hollow guidewire of claim 16 wherein at least a portion of the proximal portion is solid walled and tubular shaped.

18. The hollow guidewire of claim 3 wherein a pitch of the helical windings on the distal portion decreases in the distal direction so that a flexibility of the distal end portion increases in the distal direction.

19. The hollow guidewire of claim 16 wherein the guidewire body has a largest radial dimension between about 0.009 inches and about 0.035 inches.

20. The hollow guidewire of claim 16 wherein the distal end portion of the guidewire body comprises a plurality of openings or thinned portions that extend circumferentially about at least a portion of the distal end portion of the guidewire body.

21. The hollow guidewire of claim 16 comprising a radiopaque marker disposed at the distal portion of the guidewire body.

22. The hollow guidewire of claim 16 comprising a removable housing coupled to a proximal end of the proximal portion of the hollow guidewire body.

23. The hollow guidewire of claim 16 further comprising at least one pull wire extending through an axial lumen in the hypotube and coupled to the distal portion of the hypotube.

24. The hollow guidewire of claim 23 wherein the pull wire(s) comprise a curved surface that substantially corresponds to a shape of an inner surface of the axial lumen.

25. The hollow guidewire of claim 16 comprising a tissue removal assembly fixedly or movably disposed at the distal portion of the hollow guidewire.

26. The hollow guidewire of claim 25 wherein the tissue removal assembly comprises a rotatable drive shaft that extends through an axial lumen in the hollow guidewire and extends distally beyond a distal end of the hollow guidewire.

27. A steerable guidewire comprising:
a hollow guidewire body comprising a proximal end, a distal end, and an axial lumen that extends to the distal end;
a tissue removal assembly, wherein a portion of the tissue removal assembly is positioned at or near the distal end of the guidewire body; and
at least one pull wire that extends through the axial lumen and is coupled at or near the distal end of the hollow guidewire body, wherein a proximal force on the pull wire steers the distal end of the hollow guidewire.

28. The steerable guidewire of claim 27 wherein at least a portion of the tissue removal assembly is fixedly disposed at the distal end of the guidewire body.

29. The steerable guidewire of claim 27 wherein the tissue removal assembly is moveable from a first position in which the tissue removal assembly is disposed within the axial lumen, and a second position in which the tissue removal assembly is positioned beyond the distal end of the guidewire body.

30. The steerable guidewire of claim 27 wherein the tissue removal assembly comprises a laser, RF electrode, a resistive element, or an ultrasound transducer.

31. The steerable guidewire of claim 27 wherein the tissue removal assembly comprises a rotatable drive shaft that comprises a distal tip that extends beyond the distal end of the guidewire body.

32. The steerable guidewire of claim 27 wherein the hollow guidewire body comprises a solid tubular proximal portion integrally attached with a distal end portion, wherein the distal end portion comprises helical windings formed thereon.

33. The steerable guidewire of claim 32 wherein the guidewire body comprises a single hypotube.

34. The steerable guidewire of claim 33 wherein a pitch of the helical winding decreases in a distal direction so that a flexibility of distal end portion increases in the distal direction.

35. The steerable guidewire of claim 27 wherein the guidewire body has a largest radial dimension between about 0.009 inches and about 0.035 inches.

36. The steerable guidewire of claim 27 wherein a distal end portion of the guidewire body comprises a plurality of openings or thinned portions that extend circumferentially about at least a portion of the distal end portion of the guidewire body.

37. The steerable guidewire of claim 27 comprising a radiopaque marker disposed at the distal end of the guidewire body.

38. The steerable guidewire of claim 27 wherein the pull wire(s) comprising a curved surface that substantially corresponds to a shape of an inner surface of the axial lumen.

39. The steerable guidewire of claim 27 wherein the pull wire further comprises a substantially flat surface that faces a portion of the tissue removal assembly that extends through the axial lumen.

40. The steerable guidewire of claim 27 wherein at least one of the pull wire(s) and the tissue removal assembly are at least partially coated with Teflon.

41. The steerable guidewire of claim 27 comprising a removable housing coupled to a proximal portion of the hollow guidewire body.

42. A hollow guidewire comprising a proximal portion and a distal portion, wherein at least a part of the distal portion comprises helical windings that have a decreasing pitch between adjacent windings in the distal direction so that a distal end of the hollow guidewire is more flexible than the proximal portion of the hollow guidewire.
